(19) United States
(12) Patent Application Publication
(10) Pub. No.: US 2022/0243230 A1
(43) Pub. Date: Aug. 4, 2022

(54) BIOCONVERSION OF 4-COUMARIC ACID TO RESVERATROL

(71) Applicant: Conagen Inc., Bedford, MA (US)

(72) Inventors: Grace Park, Acton, MA (US); Ernesto Simon, Woburn, MA (US)

(73) Assignee: Conagen Inc., Bedford, MA (US)

(21) Appl. No.: 17/726,387

(22) Filed: Apr. 21, 2022

(51) Int. Cl. C12P 7/22; C12N 9/00; C12N 15/52; C12N 15/81

(52) U.S. Cl. CPC C12P 7/22; C12N 15/81; C12N 15/52; C12N 9/93

(57) ABSTRACT

The present invention relates, at least in part, to the production of resveratrol from 4-coumaric acid. The production can be mediated in a transgenic Saccharomyces cell.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Apr. 21, 2022, is named C149770089US00-SEQ-ZJG and is 89,655 bytes in size.

### FIELD OF THE INVENTION

The present disclosure generally relates to methods and materials for the conversion of 4-coumaric acid (or 3,4,5-trihydroxystilbene, also commonly known as p-coumaric acid) to resveratrol in yeasts of the Saccharomyces genus such as S. cerevisiae. In certain aspects, the present invention relates to the discovery of several transgenic strains capable of converting 4-coumaric acid to resveratrol.

### BACKGROUND OF THE INVENTION

Resveratrol is a phytophenol belonging to the group of stilbene phytoalexins, which are low-molecular-mass secondary metabolites that constitute the active defense mechanism in plants in response to infections or other stress-related events. Stilbene phytoalexins contain the stilbene skeleton (trans-1,2-diphenylethylene) as their common basic structure: that may be supplemented by addition of other groups as well. Stilbenes have been found in certain tree species (angiosperms, gymnosperms), but also in some herbaceous plants (in species of the Myrtaceae, Vitaceae and Leguminosae families). Said compounds are toxic to pests, especially to fungi, bacteria and insects. Only few plants have the ability to synthesize stilbenes, or to produce them in an amount that imparts sufficient resistance to pests.

The synthesis of the basic stilbene skeleton is pursued by stilbene synthases. Substrates that are used by known stilbene synthases include malonyl-CoA, cinnamoyl-CoA or coumaroyl-CoA. These substances occur in every plant because they are used in the biosynthesis of other important plant constituents as well such as flavonoids, flower pigments, and lipids. Resveratrol (FIG. 5, trans isomer) consists of two closely connected phenol rings and belongs therefore to the polyphenols. While present in other plants, such as eucalyptus, spruce, and lily, and in other foods such as mulberries and peanuts, resveratrol's most abundant natural sources are Vitis vinifera, -labrusca, and -muscadine (rotundifolia) grapes, which are used to make wines. The compound occurs in the vines, roots, seeds, and stalks, but its highest concentration is in the skin, which contains about 50-100 μg/g. During red wine vinification the grape skins are included in the must, in contrast to white wine vinification, and therefore resveratrol is found in small quantities in red wine only. Resveratrol has, besides its antifungal properties, been recognized for its cardioprotective and cancer chemopreventive activities; it acts as a phytoestrogen, an inhibitor of platelet aggregation, and an antioxidant. Recently it has been shown that resveratrol can also activate the SIR2 gene in yeast and the analogous human gene SIRT1, which both play a key role in extending life span. Ever since, attention is very much focused on the life-span extending properties of resveratrol.

Traditional production processes rely mostly upon extraction of resveratrol, either from the skin of grape berries, or from knotweed. This is a labor-intensive process and generates low yield which, therefore, prompts an incentive for the development of novel, more efficient and high-yielding production processes.

In plants, the phenylpropanoid pathway is responsible for the synthesis of a wide variety of secondary metabolic compounds, including lignins, salicylates, coumarins, hydroxycinnamic amides, pigments, flavonoids and phytoalexins. Indeed, formation of resveratrol in plants proceeds through the phenylpropanoid pathway. The amino acid L-phenylalanine is converted into trans-cinnamic acid through the non-oxidative deamination by L-phenylalanine ammonia lyase (PAL) (FIG. 6). Next, trans-cinnamic acid is hydroxylated at the para-position to 4-coumaric acid (4-hydroxycinnamic acid, also commonly known as p-coumaric acid) by cinnamate-4-hydroxylase (C4H), a cytochrome P450 monooxygenase enzyme, in conjunction with NADPH:cytochrome P450 reductase (CPR). The 4-coumaric acid is subsequently activated to 4-coumaroyl-CoA by the action of 4-coumarate:CoA ligase (4CL). Finally, resveratrol synthase (VST) catalyzes the condensation of a phenylpropane unit of 4-coumaroyl-CoA with malonyl CoA, resulting in formation of resveratrol.

A yeast was disclosed that was able to produce resveratrol from 4-coumaric acid that is found in small quantities in grape must (Becker et al.). The production of 4-coumaroyl-CoA, and concomitant resveratrol, in laboratory strains of S. cerevisiae, was achieved by co-expressing a heterologous coenzyme-A ligase gene, from hybrid poplar, together with the grapevine resveratrol synthase gene (vst1). The other substrate for resveratrol synthase, malonyl-CoA, is already endogenously produced in yeast and is involved in de novo fatty-acid biosynthesis. The study showed that cells of S. cerevisiae could produce minute amounts of resveratrol, either in the free form or in the glucoside-bound form, when cultured in synthetic medium that was supplemented with 4-coumaric acid.

However, said yeast would not be suitable for a commercial application because it suffers from low resveratrol yield.

### SUMMARY OF THE INVENTION

In a first aspect, provided herein is a microorganism of the genus Saccharomyces, comprising a disrupted gene encoding an enzyme involved in the degradation of phenylpyruvate to phenylacetaldehyde. In a set of embodiments, the disrupted gene encoding an enzyme involved in the degradation of phenylpyruvate to phenylacetaldehyde is selected from the group consisting of ARO10, PDC5, and combinations thereof. The gene encoding an enzyme involved in the degradation of phenylpyruvate to phenylacetaldehyde may be disrupted by partial or total deletion. The microorganism may further comprise a recombinant gene encoding a 4-coumaric acid:Coenzyme A ligase from Arabidopsis thaliana. An exemplary recombinant gene encoding a 4-coumaric acid:Coenzyme A ligase from Arabidopsis thaliana is At4CL1. In one embodiment, the recombinant gene encoding a 4-coumaric acid:Coenzyme A ligase has at least 90%, 95%, or 99% sequence identity to any one of SEQ. ID. NOs: 1 and 12. In a further embodiment, the recombinant gene encoding a 4-coumaric acid:Coenzyme A ligase has at least 98% or 99% sequence identity to any one of SEQ. ID. NOs: 1 and 12. In an additional embodiment, the recombinant gene encoding a 4-coumaric acid:Coenzyme A ligase has a sequence according to any one of SEQ. ID. NOs: 1 and 12. The microorganism may also further comprise a recombinant gene encoding a Vitis vinifera stilbene synthase. In one embodiment, the Vitis vinifera stilbene synthase gene has at least 90%, 95%, or 99% sequence identity to any one of SEQ. ID. NOs.: 3, 4, 5, 6, 7, and 8. In a further embodiment, the Vitis vinifera stilbene synthase gene has at least 98%, or 99% sequence identity to any one of SEQ. ID. NOs.: 3, 4, 5, 6, 7, and 8. In an additional embodiment, the Vitis vinifera stilbene synthase gene has a nucleotide sequence selected from the group consisting of SEQ. ID. NOs.: 3, 4, 5, 6, 7, and 8. In addition to the foregoing, the microorganism may comprise a recombinant gene encoding a feedback inhibition-resistant mutant of an acetyl-CoA carboxylase. In a first embodiment, the gene encoding a feedback inhibition-resistant mutant of an acetyl-CoA carboxylase has at least 90%, 95%, or 99% sequence identity to SEQ. ID. NO: 10. In a second embodiment, the gene encoding a feedback inhibition-resistant mutant of an acetyl-CoA carboxylase has at least 98%, or 99% sequence identity to SEQ. ID. NO: 10. In a third embodiment, the gene encoding a feedback inhibition-resistant mutant of an acetyl-CoA carboxylase comprises a nucleotide sequence according to SEQ ID NO: 10. In a fourth embodiment, the feedback inhibition-resistant mutant of an acetyl-CoA carboxylase comprises an amino acid sequence according to SEQ ID NO: 11. As stated above, the microorganism is a yeast of the genus Saccharomyces. In a preferred embodiment, the microorganism is of the species Saccharomyces cerevisiae.

In a second aspect, provided herein is a method of producing resveratrol using a recombinant Saccharomyces cell, the method comprising: (i) cultivating a recombinant Saccharomyces cell in a medium; (ii) adding 4-coumaric acid to the medium to initiate the bioconversion of 4 coumaric acid to resveratrol; and (iii) extracting resveratrol from at least one of the recombinant cell and medium, wherein the recombinant Saccharomyces cell has been transformed to disrupt a gene encoding an enzyme involved in the degradation of phenylpyruvate to phenylacetaldehyde. In representative embodiments, the disrupted gene encoding an enzyme involved in the degradation of phenylpyruvate to phenylacetaldehyde is selected from the group consisting of ARO10, PDC5, and combinations thereof. The Saccharomyces cell may have been further transformed with a nucleic acid construct encoding a 4-coumaric acid:Coenzyme A ligase from Arabidopsis thaliana. In one, non-limiting embodiment, the 4-coumaric acid:Coenzyme A ligase from Arabidopsis thaliana comprises an amino acid sequence according to SEQ ID NO: 2. The Saccharomyces cell may be also transformed with a nucleic acid construct encoding a stilbene synthase from Vitis vinifera. In one exemplary embodiment, the stilbene synthase from Vitis vinifera comprises an amino sequence according to SEQ ID NO: 9. In addition to the foregoing, the Saccharomyces cell may have been further transformed with a nucleic acid construct encoding a feedback inhibition-resistant mutant of an acetyl-CoA carboxylase. In a representative embodiment, the feedback inhibition-resistant mutant of an acetyl-CoA carboxylase comprises an amino acid sequence according to SEQ ID NO: 11. As stated above, the microorganism is a yeast of the genus Saccharomyces. In a preferred embodiment, the microorganism is of the species Saccharomyces cerevisiae.

Resveratrol produced using the methods and/or the isolated recombinant host cells described herein can be collected and incorporated into a consumer product. For example, the resveratrol can be admixed with a consumer product. In some embodiments, the resveratrol can be incorporated into the consumer product in an amount sufficient to impart, modify, boost or enhance a ______.

Other features and advantages of the present invention will become apparent in the following detailed description, taken with reference to the accompanying drawings.

### BRIEF DESCRIPTION OF THE DRAWINGS

FIG. 1 is a bar graph illustrating the effect of 4-coumaric acid at different concentrations on yeast cultures.

FIG. 2 is a bar graph illustrating resveratrol production, phloretic acid production, and amounts of leftover 4-coumaric acid from Generation 1 strains. RSV: resveratrol; PA: phloretic acid; pCA: 4-coumaric acid.

FIG. 3 is a bar graph illustrating resveratrol and phloretic acid production from Generation 1 strains. Parent strain: Generation 1 strains; Cured: cured Generation 1 strains; Set 4.30 and set 4.31: transformants of set 4.30 or 4.31 (see Table 1); VvSTS in 2u plasmid; strains containing VvSTS expression cassettes in a 2u plasmid.

FIG. 4 is a bar graph illustrating resveratrol and phloretic acid production from Generation 3 strains. Parent strain: Generation 1 or 2 strains; Set 7.23 FDC1/PAD1 KO; FDC1 and PAD1 knocked out strains; Acc1 fbr int.: Acc1 feedback inhibition resistant mutant integrated strains; FDC1/PAD1 KO+ACC1 fbr int; double mutant of FDC1 and PAD1 knock-out plus Acc1 feedback inhibition resistant mutant integrated strains.

FIG. 5 shows the chemical structure of trans-resveratrol.

FIG. 6 illustrates the phenylpropanoid pathway utilizing phenylalanine ammonia lyase on L-phenylalanine.

### DETAILED DESCRIPTION

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of proteins. It includes bacteria, yeast, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art and is used without limitation to refer to a DNA sequence that encodes a specific amino acid sequence.

“Growing” or “cultivating” a cellular system includes providing an appropriate medium that would allow cells to multiply and divide. It also includes providing resources so that cells or cellular components can translate and make recombinant proteins.

“Yeasts” are eukaryotic, single-celled microorganisms classified as members of the fungus kingdom. Yeasts are unicellular organisms which evolved from multicellular ancestors but with some species useful for the current invention being those that have the ability to develop multicellular characteristics by forming strings of connected budding cells known as pseudo hyphae or false hyphae.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. In any one embodiments provided herein, a particular nucleic acid sequence can also encompass conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature.

An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing resveratrol.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,‘ and “peptide” are to be given their respective ordinary’ and customary meanings to a person of ordinary skill in the art; the three terms are sometimes used interchangeably and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction). In any one embodiment, the AghSHC1 polypeptide may be a functional fragment.

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide. In any one embodiment, the AghSHC1 polypeptide may be a functional variant.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide. In any one embodiment, the AghSHC1 polypeptide may be a variant with any one of the foregoing percentage identities. Preferably such a AghSHC1 polypeptide is functional in the conversion of 4-coumaric acid to resveratrol.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology or production of a gene product in transgenic, transformed or recombinant organisms.

“Transformation” is to be given its ordinary and customary meaning to a person of ordinary skill in the art and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed” or “recombinant”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

“Protein Expression” refers to protein production that occurs after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity genes of the current invention are useful in the production of resveratrol as provided herein and have a substantial percent sequence identity to the polynucleotide sequences provided herein and are encompassed within the scope of this invention.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As used herein, the term “disrupted gene” refers to a gene containing one or more mutations (e.g., insertion, full or partial deletion, or full or partial nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express a detectable level (e.g. by enzymatic activity) of the protein encoded by the gene. A cell that does not express a detectable level of the protein may be referred to as a knockout cell. For example, a cell having an ARO10 gene edit may be considered a knockout cell if enzymatic activity associated with the protein cannot be detected using a substrate specific for the ARO10 enzyme.

### Constructs According to the Present Invention

In some aspects, the present invention relates to constructs like expression vectors for expressing a transgenic polypeptide.

In an embodiment, the expression vector includes those genetic elements for expression of a recombinant polypeptide described herein (e.g., a 4-coumaric acid:Coenzyme A ligase) in various host cells. The elements for transcription and translation in the host cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR). In molecular cloning, a vector is a DNA molecule used as a vehicle to artificially carry foreign genetic material into another cell, where it can be replicated and/or expressed (e.g. plasmid, cosmid, Lambda phages). A vector containing foreign DNA is considered recombinant DNA. The four major types of traditional vectors are plasmids, viral vectors, cosmids, and artificial chromosomes. Of these, the most commonly used vectors are plasmids. Common to all engineered vectors are an origin of replication, a multicloning site, and a selectable marker.

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun et al, BIOTECHNIQUES 13, 515-18 (1992), each of which are incorporated herein by reference).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared using PCR appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

In some embodiments, the transformed cell is a plant cell, an algal cell, a fungal cell, or a yeast cell of the Saccharomyces genus, e.g., Saccharomyces cerevisiae.

Microbial host cell expression systems and expression vectors containing regulatory sequences that direct high-level expression of foreign proteins that are well-known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Termination control regions may also be derived from various genes native to the microbial hosts. A termination site optionally may be included for the microbial hosts described herein.

Preferred host cells include those known to have the ability to produce resveratrol from 4-coumaric acid. For example, preferred host cells can include yeast of the species Saccharomyces cerevisiae.

### Recombinant Saccharomyces Strains

Converting 4-coumaric acid to resveratrol requires two reaction steps including the ligation of Coenzyme A to 4-coumaric acid and the condensation of one mole of coumaroyl-CoA and three moles of malonyl-CoA. The inventors have engineered host Saccharomyces strains to convert 4-coumaric acid to resveratrol by integrating expression cassettes of a 4-coumaroyl-CoA (4CL) ligase from Arabidopsis thaliana and expression cassettes of a stilbene synthase from Vitis vinifera (VvSTS). To increase malonyl-CoA supply, the inventors have also integrated overexpression cassettes of feedback inhibition-resistant mutant acetyl-CoA carboxylase (ACC1). By engineering a host cell as provided herein and cultivating the engineered host strain in a mixture including 4-coumaric acid, the inventors were able to achieve high levels of resveratrol production.

The Saccharomyces strains of this aspect of the invention have been transformed to disrupt one or more genes encoding native enzymes that are involved in the degradation of phenylpyruvate. Without being bound to any particular theory, it is believed that this transformation improves resveratrol production by eliminating competing pathways for the precursor phenylpyruvate. One such gene is that coding for ARO10, a phenylpyruvate decarboxylase that catalyzes the decarboxylation of phenylpyruvate to phenylacetaldehyde. PDC5 is another phenylpyruvate decarboxylase native to Saccharomyces. As such, in a representative embodiment, either or both Saccharomyces cerevisiae genes ARO10 (SEQ ID NO: 21) and PDC5 (SEQ ID NO: 22) may be disrupted by any of the methods outlined above. In an exemplary embodiment, both genes are disrupted by partial or total sequence deletion.

Four At4CL genes have been identified in Arabidopsis thaliana (At4CL1-At4CL4), any of which may be transformed into a Saccharomyces species such as S. cerevisiae. In a non-limiting embodiment, the Saccharomyces strain is transformed to express a gene coding for At4CL1 (SEQ ID NO: 2), a gene coding for At4CL2 (SEQ ID NO: 13), or both. The 4CL gene or genes may be codon optimized or harmonized, as is the case for the sequences according to SEQ. ID. NOs: 1 and 12. In one embodiment, the recombinant 4CL gene has at least 90%, 95%, or 99% sequence identity to any one of SEQ. ID. NOs: 1 and 12. In another embodiment, the recombinant 4CL gene has at least 98% or 99% sequence identity to any one of SEQ. ID. NOs: 1 and 12.

To enhance bioconversion efficiency, the Saccharomyces strain may be transformed to host multiple copies of a gene encoding a stilbene synthase from Vitis vinifera (VvSTS). In representative embodiments, the number of VvSTS genes that are transformed into the host cell may be 2, 3, 4, or 5. Each gene may be selected from a number of differently codon optimized versions of VvSTS, such as those according to sequences SEQ ID NOs: 3, 4, 5, 6, 7, and 8. In one embodiment, each VvSTS gene has at least 90%, 95%, or 99% sequence identity to any one of SEQ. ID. NOs.: 3, 4, 5, 6, 7, and 8. In a further embodiment, each VvSTS gene has at least 98%, or 99% sequence identity to any one of SEQ. ID. NOs.: 3, 4, 5, 6, 7, and 8. In an additional embodiment, each VvSTS gene has a nucleotide sequence selected from the group consisting of SEQ. ID. NOs.: 3, 4, 5, 6, 7, and 8.

Acetyl-CoA carboxylase (ACC) is a biotin-dependent enzyme that catalyzes the carboxylation of acetyl-CoA to produce malonyl-CoA. This enzyme is rate-limiting for the biosynthesis of fatty acids and is known to be inhibited by phosphorylation. Therefore, in some embodiments, the host strain is transformed with a recombinant gene coding for a feedback-inhibition resistant mutant of the S. cerevisiae ACC1 enzyme. In the example mutant of SEQ ID NO: 11, two amino acid substitutions occur at position 659 and 1157, where serine residues have been changed to alanines. In one embodiment, the gene encoding a feedback inhibition-resistant mutant of an acetyl-CoA carboxylase has at least 90%, 95%, or 99% sequence identity to SEQ. ID. NO: 10. In a further embodiment, the gene encoding a feedback inhibition-resistant mutant of an acetyl-CoA carboxylase comprises a nucleotide sequence according to SEQ ID NO: 10. In an additional embodiment, the gene encoding a feedback inhibition-resistant mutant of an acetyl-CoA carboxylase comprises a nucleotide sequence according to SEQ ID NO: 10.

### Production of Resveratrol

In a further aspect, provided herein is a method for producing resveratrol using a recombinant cell as exemplified by the aforesaid recombinant Saccharomyces strains. A recombinant Saccharomyces host cell, e.g., Saccharomyces cerevisiae, is cultivated in a medium, and 4-coumaric acid is added to the medium to initiate its bioconversion to resveratrol which is then extracted from at least one of the recombinant cell and medium. The recombinant Saccharomyces cell has been transformed to disrupt a gene encoding an enzyme involved in the degradation of phenylpyruvate to phenylacetaldehyde. In one representative embodiment, the disrupted gene may one or both of ARO10 and PDCS.

In some embodiments, the host cell has been further transformed with a nucleic acid encoding a 4-coumaric acid:Coenzyme A ligase from Arabidopsis thaliana. In one non-limiting example, the 4-coumaric acid:Coenzyme A ligase from Arabidopsis thaliana comprises an amino acid sequence according to SEQ ID NO: 2. In more embodiments, the host cell has been further transformed with a nucleic acid construct encoding a stilbene synthase from Vitis vinifera. In one non-limiting example, the stilbene synthase from Vitis vinifera comprises an amino sequence according to SEQ ID NO: 9. In representative embodiments, the number of VvSTS genes that are transformed into the host cell may be 2, 3, 4, or 5. Each gene may be selected from a number of differently codon optimized versions of VvSTS, such as those according to sequences SEQ ID NOs: 3, 4, 5, 6, 7, and 8. In one embodiment, each VvSTS gene has at least 90%, 95%, or 99% sequence identity to any one of SEQ. ID. NOs.: 3, 4, 5, 6, 7, and 8. In a further embodiment, each VvSTS gene has at least 98%, or 99% sequence identity to any one of SEQ. ID. NOs.: 3, 4, 5, 6, 7, and 8. In an additional embodiment, each VvSTS gene has a nucleotide sequence selected from the group consisting of SEQ. ID. NOs.: 3, 4, 5, 6, 7, and 8. In one embodiment, the stilbene synthase from Vitis vinifera comprises an amino sequence according to SEQ ID NO: 9. In a set of additional embodiments, the host cell has been further transformed with a nucleic acid construct encoding a feedback inhibition-resistant mutant of an acetyl-CoA carboxylase. In a representative example, the feedback inhibition-resistant mutant of an acetyl-CoA carboxylase comprises an amino acid sequence according to SEQ ID NO: 11.

Cultivation of host cells can be carried out in an aqueous medium in the presence of usual nutrient substances. A suitable culture medium, for example, can contain a carbon source, an organic or inorganic nitrogen source, inorganic salts and growth factors. For the culture medium, glucose can be a preferred carbon source. Phosphates, growth factors and trace elements can be added.

An illustrative example of a production process is provided in the Examples.

One skilled in the art will recognize that the resveratrol composition produced by such methods can be further purified and mixed with the ingredients of edible consumer products as described above.

The disclosure will be more fully understood upon consideration of the following non-limiting Examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Example 1

### Construction of Background Strains for Bioconversion Process

The genome of S. cerevisiae strain BY4741 was modified by deletion of the Aro10 open reading frame. Aro10 is phenylpyruvate decarboxylase catalyzing phenylpyruvate degradation to phenylacetaldehyde. The gene was deleted by replacing Aro10 with the Met15 marker. Approximately 1000 base pairs of upstream and downstream flanking regions of Aro10 coding sequences were amplified producing two PCR products. A complete gene sequence of Met15 including promoter and terminator was amplified separately. Those three PCR products, Aro10 upstream, Met15 and Aro10 downstream were stitched together by overlapping PCR to produce an Aro10 knock out DNA fragment. The DNA fragment was transformed directly into the BY4741 strain and selected for methionine prototrophy. The resulting strain was designated as CNFS004 and was used as a background strain for all resveratrol bioconversion strains.

The CNFS004 strain was further modified by integrating At4CL1, one of the resveratrol biosynthetic pathway genes. At4CL1 is one of four 4-coumaric acid:Coenzyme A ligase from Arabidopsis thaliana. The open reading frame of At4CL1 was codon optimized (SEQ ID NOS:1 and 2) and integrated into the PDC5 locus. PDC5 is another decarboxylase that degrades phenylpyruvate. The entire open reading frame of PDC5 was replaced with At4CL1 flanked by the PGK1 promoter and the SSA1 terminator. The product strain was designated as CNFS007.

CNFS007 was subjected to the integration of five copies of differently codon-optimized Vitis vinifera stilbene synthases (SEQ ID NO: 3, 4, 5, 6, 7, 8 and 9) and one copy of a gene coding for a feedback inhibition-resistant mutant of the native Saccharomyces cerevisiae acetyl-CoA carboxylase ACC1 (SEQ ID NO: 10). ACC1 is a rate-limiting enzyme for fatty acid biosynthesis which is known to be inhibited by phosphorylation. Therefore, the strain was transformed to express the feedback-resistant mutant having the amino acid sequence of SEQ ID NO: 11. The mutant contains two amino acid changes at position 659 and 1157, where two serine residues were changed to alanine. The resulting strains were designated as CNFS109, CNFS110, CNFS111, CNFS112 and CNFS113. These strains were genetically identical but selected as independent isolates.

### Example 2

### Construction of Generation 1 Strains for Bioconversion Process

The strains of Example 1 were tested in microcultures with 500 mg/L of 4-coumaric acid fed 24 hours post inoculation. CNFS109 and CNFS110 were inoculated into 50 ml of synthetic drop out medium without uracil (Sigma-Aldrich, St. Louis, Missouri) and incubated overnight under shaking conditions. Saturated culture was dispensed into individual wells in 24 well plates that contained 2 ml of synthetic drop out medium without uracil. 4-coumaric acid dissolved in acidified ethanol (20 g/L stock, in ethanol : water : HCl=50:49:1) was added to each well, to form media having 4-coumaric concentrations of 0 mg/L, 100 mg/L, 500 mg/L, 1000 mg/L, and 2000 mg/L, respectively. All experiments were performed in triplicate. The cultures were incubated under shaking for 48 hours (250 rpm, 30 ° C.).

Resveratrol was extracted by adding equal volumes of methanol. The samples were analyzed by high performance liquid chromatography using an Avantor ACE Excel 2 C18-PFP column (150×2.1 mm). The chromatography was operated using Thermo Scientific Vanquish system. Mobile phase A was 0.1% trifluoroacetic acid in water and mobile phase B was 100% acetonitrile. The chromatography was performed using linear gradient method with a 0.3 ml/minute flow rate, i.e., 0 minutes to 2 minutes 10% for B, linear gradient for 2 to 7 minutes 10% to 60% of B, then maintained % of B for three minutes, and prime the column by 10% of B for 1 minute. Eluted compounds were detected by diode array illumination at the UV wavelength of 280 nm.

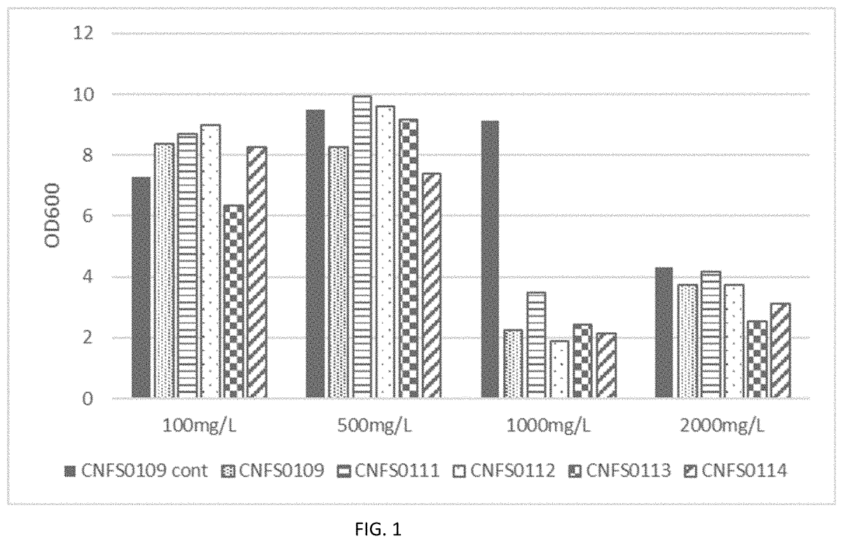

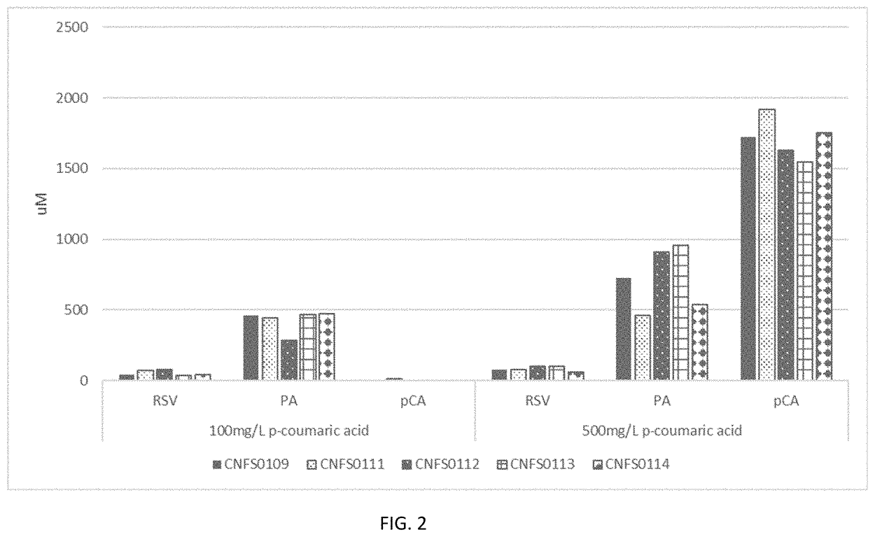

The bar graph of FIG. 1 illustrates the effect of 4-coumaric acid at different concentrations on yeast cultures. There was little or no impact due to 4-coumaric acid toxicity at concentration of up to 500 mg/L, but the survival rate declined when 1 g/L or 2 g/L of 4-coumaric acid were added to the culture. Equivalent amounts of ethanol added in the absence of 4-coumaric acid did not decrease cell growth at concentrations up to 10% of culture volume (FIG. 1, CNFS109 cont.). Due to the toxicity associated with high concentrations of 4-coumaric acid which inhibited yeast from growing and metabolizing, resveratrol bioconversion was only observed when 4-coumaric was added to the yeast culture at relatively lower concentrations (FIG. 2).

When 4-coumaric acid was added at a concentration of 100 mg/L of, most of 4-coumaric acid was consumed, but the majority of the conversion was to phloretic acid (˜90%). Phloretic acid is produced by native Saccharomyces TSC13, an enzyme having double bond reductase activity on long chain fatty acids. It has been reported that coumaroyl-CoA is the substrate for TSC13 (Lehka et al (FEMS yeast research 17, 2017). When 500 mg/L of 4-coumaric acid was fed, only half of the 4-coumaric acid was converted to phloretic acid (˜32%, g/g) and resveratrol (˜10%, g/g).

### Example 3

### Construction of Generation 2 Strains for Bioconversion Process

Strains CNFS109, CNFS111, CNFS112 and CNFS113 were further modified to improve resveratrol bioconversion yields by integrating another multiple copy of Vitis vinifera stilbene synthase. In order to integrate another integration cassette using a uracil marker, CNFS109 was cured to CNFS113 by growing the strains on 5-FOA (5-fluoroorotic acid) plate. The resulting strains were designated as CNFS261, CNFS262, CNFS263 and CNFS264, respectively. These cured strains were engineered to integrate an At4CL2 gene (SEQ ID NO: 12) with or without a copy of feedback inhibition-resistant mutant of SeACS1 (SEQ ID NO:15 and 16), an acetyl CoA synthetase from Salmonella enterica acetyl CoA synthetase. One set of integration cassettes (set 4.30, Table 1) contains four copies of differently codon optimized VvSTS (SEQ ID NO: 3, 4, 5, 6, 7, 9, one copy of At4CL2, and one copy of SeACS1 whose amino acid reside on leucine 641 was mutated to proline (Starai et al.) (Table 1). The other set of integration cassettes (set 4.31, Table 1) contained five copies of VvSTS (SEQ ID NO: 3, 4, 5, 6, 7, 8, 9) and one copy of At4CL2 (SEQ ID NO: 12). The strains were also transformed with 2u plasmid harboring a VvSTS expression cassette (SEQ ID NO:14) (Table 1). For unknown reasons, set 4.30 transformants were only attained on CNFS113 background strain. No transformants of set 4.31 on CNFS111 background strain could be obtained.

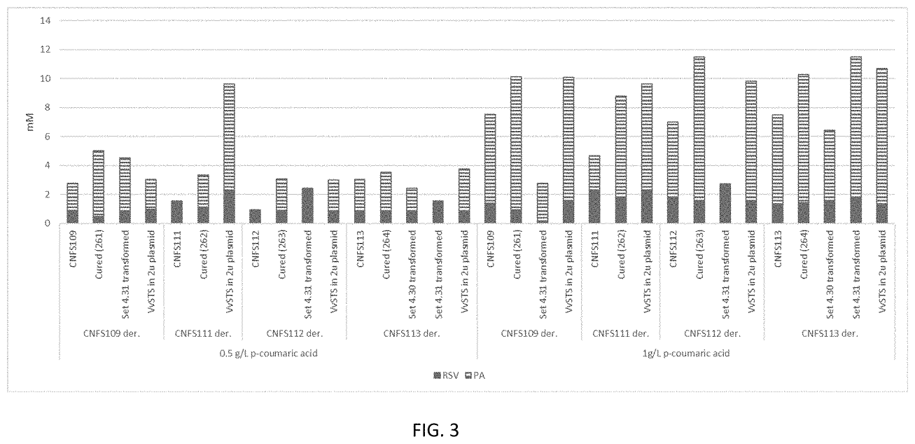

Several isolates from each transformation were tested in microculture. A number of colonies were picked from the transformation plate and inoculated on a 96-well microculture plate. After 48 hours of incubation at 30° C. to make the culture reach saturation, 80 μl of seed culture were inoculated into 48-well plates containing 1 ml of fermentation medium. The medium was composed of synthetic drop out medium without uracil buffered by 50 mM succinate (pH 6.0) with addition of 40 g/L EnPump (Enpresso GmbH, Berlin, Germany), 0.4% reagent A, 2% vitamin solution (50 mg biotin, 200 mg p-aminobenzoic acid, 1 g nicotinic acid, 1 g Ca-pantothenate, 1 g pyridoxine-HCl, 1 g thiamine-HCl, and 25 g myo-inositol per liter) and 2% yeast extract. After 62 hours of culture 0.5 g/L or 1 g/L of 4-coumaric acid was added. The bioconversion products were extracted and analyzed using the same method described in Example 2. FIG. 3 reports the results of microculture screening. Most transformants produced similar or reduced amount of resveratrol as compared to parent strains, but the transformants of CNFS263 (i.e., the CNFS112 derivative) with set 4.31 (five copies of VvSTS and one copy of At4CL2) exhibited increased resveratrol production reaching a 70% conversion rate and did not produce phloretic acid. This strain was designated as CNFS283.

### Example 4

### Construction of Generation 3 Strain for Bioconversion Process

To enhance bioconversion efficiency, CNFS283 and the previous best strain CNFS111 (FIG. 3) were subjected to another round of transformation. This time, not only integration cassettes containing VvSTS and a copy of feedback inhibition mutant of Saccharomyces cerevisiae ACC1, ScACC1_{S659A, S1157A} (set 7.23, Table 1), but also FDC1/PAD1 knock-out cassette to disrupt cinnamic acid decarboxylase and p-coumaric acid degradation, as well as a copy of ScACC1_{S659A, S1157A} overexpression cassette were transformed into the strains.

TABLE 1

| | | | assembler 1 | | assembler 2 | | assembler 3 | |
|---|---|---|---|---|---|---|---|---|
| | location | set | ORF1 | ORF2 | ORF1 | ORF2 | ORF1 | ORF2 |
| Generation 1 | XII-5 | Set 5.1 | VvSTS opt2 | VvSTS opt5 | VvSTSopt1 | VvSTS opt3 | Acc1-fbr | VvSTSopt4 |
| Generation 2 | XI-2 | Set 4.30 | VvSTS opt2 | VvSTS opt5 | At4CL2-opt | VvSTSopt3 | SeACS1_{L641P} | VvSTSopt4 |
| | | Set 4.31 | VvSTS opt2 | VvSTS opt5 | At4CL2-opt | VvSTSopt3 | VvSTS-opt6 | VvSTSopt4 |
| Generation 3 | XI-3 | Set 7.23 | VvSTS opt2 | VvSTS opt5 | VvSTSopt1 | VvSTSopt3 | pADH1-ScACC1_{S659A, S1157A} | VvSTSopt4 |

| | | |
|---|---|---|
| XI-3 | N.A. | XI-3::pADH1-ScACC1_{S659A, S1157A}, ZeoR |
| FDC1/PAD1 | N.A. | ΔFDC1ΔPAD1::ZeoR |

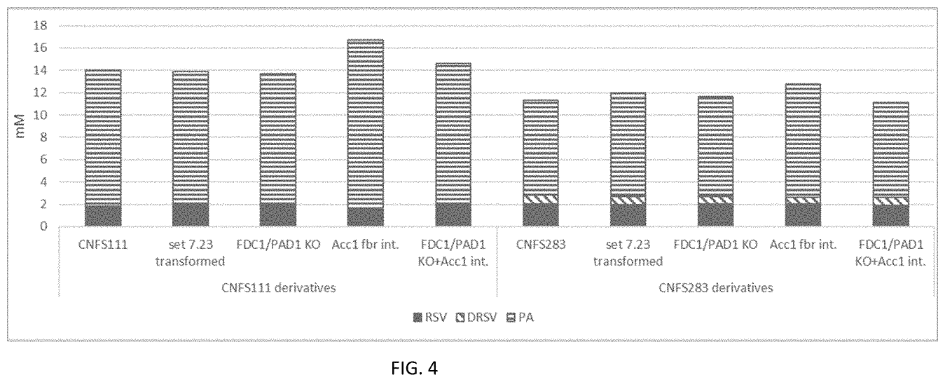

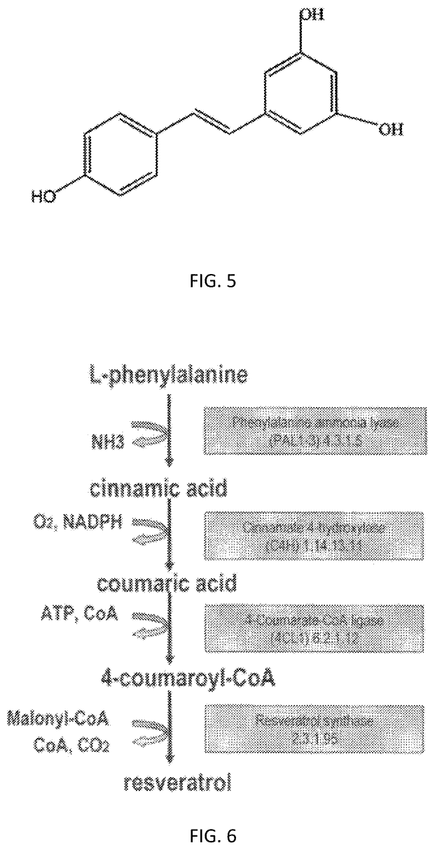

Several transformants were picked from each plate and inoculated in a 96-well microculture plate. Isolates were tested in the same conditions as previously described in Example 3 except that 1 g/L of 4-coumaric acid was added to account for the expected increase in substrate demand in view of the larger number of stilbene synthase genes. However, and unexpectedly, increasing the gene copy number of stilbene synthase and ScACC1_{S659A, S1157A} boosted production of resveratrol only slightly (FIG. 4). Instead, an increase in dihydroresveratrol production was found (FIG. 4). Dihydroresveratrol is a by-product of the resveratrol biosynthesis pathway. According to Eichenberger et al (2017), it is speculated that coumaroyl-CoA is converted to dihydrocoumaroyl-CoA by ScTSC13. The molecule, in turn, become substrates for stilbene synthase, thereby generating dihydroresveratrol. The conversion percentage reached nearly 70% in the third generation strains when the dihydroresveratrol product was accounted for. Phloretic acid production was reduced in the third generation strains, which suggested that phloretic acid is converted to dihydroresveratrol (FIG. 4). Without being bound to any particular theory, the increase in dihydroresveratrol production in the third generation strains may be attributed to the fact that stilbene synthase typically binds to coumaroyl-CoA but also promiscuously binds to dihydrocoumaroyl-CoA when the enzyme concentration is high.

### Materials and Methods

### DNA Manipulation

### Cloning and Plasmid Construction

Gibson assembly cloning was employed to assemble genes of interest, i.e. differently codon optimized VvSTS, At4CL, as well as feedback inhibition resistant mutant ACC1, to make integration vectors. Integration vectors were built to integrate multiple genes in the chromosomal locations described in Mikkelson et al. (2012 Metabolic engineering 14. P. 104-111). Three integration vectors containing two genes of interest each were prepared and transformed simultaneously. Homologous recombination via homology arms in each of the plasmids enabled to integrate all six expression cassettes into target locations. Multiple coding sequences were amplified by the Q5 PCR system prior to cloning. PDCS was knocked out by integrating a DNA fragment containing 500 base pairs from the upstream and downstream regions of the coding sequences of PDCS and At4CL and nourseothricin expression cassettes in the middle. This enabled the knocking out of PDCS and the integration of At4CL at the same time. All DNA fragments obtained by PCR were stitched together by overlapped PCR. The final PCR fragment was transformed directly into Saccharomyces cerevisiae cells. FDC1 and PAD1 knock out constructs were made by assembling 3 PCR fragments including 500 base pairs of upstream and downstream sequences of the FDC1 and PAD1 coding regions and a phleomycine expression cassette. The PCR fragments were assembled by overlapped PCR and cloned into pMiniT PCR cloning vector (NEB). The plasmid was linearized by restriction enzyme digestion prior to transformation. All reagents for Gibson assembly cloning were purchased from NEB.

### REFERENCES

Becker et al. (2003) Metabolic engineering of S. cerevisiae for the synthesis of the wine-related antioxidant resveratrol (FEMS yeast research 4 (2003) p.'79-85).
Zhang et al. (2006) Using unnatural protein fusions to engineer resveratrol biosynthesis in yeast and mammalian cells (JACS communications 128, p. 13030-13031).
Sydor et al. (2010) Considerable increase in resveratrol production by recombinant industrial yeast strains with use of rich medium. (Applied and environmental microbiology, p. 3361-3363).
Thapa et al. (2019) (Molecules 24, p. 2571) Biotechnological advances in resveratrol production and its chemical diversity.
Yuan et al. (2020) (Microbial cell factories 19. P. 143) De novo resveratrol production through modular engineering of an Escherichia coli-Saccharomyces cerevisiae co-culture.
Eichenberger et al. (2017) (Metabolic Engineering 39. P. 80) Metabolic engineering of Saccharomyces cerevisiae for de novo production of dihydrochalcones with known antioxidant, antidiabetic and sweet tasting properties.
Mikkelsen et al. (2012) (Metabolic Engineering 14. P. 104) Microbial production of indolylglucosinolate through engineering of a multi-gene pathway in a versatile yeast expression platform.
Starai et al. (2005) (J. Biol. Chem. Vol 280. NO. 28. P. 26200-26205) Residue Leu-641 of Acetyl-CoA Synthetase is Critical for the Acetylation of Residue Lys-609 by the Protein Acetyltransferase Enzyme of Salmonella enterica.

Nucleic Acid and Amino Acid Sequences
Synthetic DNA
Codon optimized Arabidopsis thaliana coumaroyl CoA ligase 1
SEQ ID NO: 1
ATGGCGCCACAAGAACAAGCAGTTTCTCAGGTGATGGAGAAACAGAGCAACAACAACAACAGTGACGTCATTT
TCCGATCAAAGTTACCGGATATTTACATCCCGAACCACCTATCTCTCCACGACTACATCTTCCAAAACATCTC
CGAATTCGCCACTAAGCCTTGCCTAATCAACGGACCAACCGGCCACGTGTACACTTACTCCGACGTCCACGTC
ATCTCCCGCCAAATCGCCGCCAATTTTCACAAACTCGGCGTTAACCAAAACGACGTCGTCATGCTCCTCCTCC
CAAACTGTCCCGAATTCGTCCTCTCTTTCCTCGCCGCCTCCTTCCGCGGCGCAACCGCCACCGCCGCAAACCC
TTTCTTCACTCCGGCGGAGATAGCTAAACAAGCCAAAGCCTCCAACACCAAACTCATAATCACCGAAGCTCGT
TACGTCGACAAAATCAAACCACTTCAAAACGACGACGGAGTAGTCATCGTCTGCATCGACGACAACGAATCCG
TGCCAATCCCTGAAGGCTGCCTCCGCTTCACCGAGTTGACTCAGTCGACAACCGAGGCATCAGAAGTCATCGA
CTCGGTGGAGATTTCACCGGACGACGTGGTGGCACTACCTTACTCCTCTGGCACGACGGGATTACCAAAAGGA
GTGATGCTGACTCACAAGGGACTAGTCACGAGCGTTGCTCAGCAAGTCGACGGCGAGAACCCGAATCTTTATT
TCCACAGCGATGACGTCATACTCTGTGTTTTGCCCATGTTTCATATCTACGCTTTGAACTCGATCATGTTGTG
TGGTCTTAGAGTTGGTGCGGCGATTCTGATAATGCCGAAGTTTGAGATCAATCTGCTATGGGAGCTGATCCAG
AGGTGTAAAGTGACGGTGGCTCCGATGGTTCCGCCGATTGTGTTGGCCATTGCGAAGTCTTCGGAAACGGAGA
AGTATGATTTGAGCTCGATAAGAGTGGTGAAATCTGGTGCTGCTCCTCTTGGTAAAGAACTTGAAGATGCCGT
TAATGCCAAGTTTCCTAATGCCAAACTCGGTCAGGGATACGGAATGACGGAAGCAGGTCCAGTGCTAGCAATG
TCGTTAGGTTTTGCAAAGGAACCTTTTCCGGTTAAGTCAGGAGCTTGTGGTACTGTTGTAAGAAATGCTGAGA
TGAAAATAGTTGATCCAGACACCGGAGATTCTCTTTCGAGGAATCAACCCGGTGAGATTTGTATTCGTGGTCA
CCAGATCATGAAAGGTTACCTCAACAATCCGGCAGCTACAGCAGAAACCATTGATAAAGACGGTTGGCTTCAT
ACTGGAGATATTGGATTGATCGATGACGATGACGAGCTTTTCATCGTTGATCGATTGAAAGAACTTATCAAGT
ATAAAGGTTTTCAGGTAGCTCCGGCTGAGCTAGAGGCTTTGCTCATCGGTCATCCTGACATTACTGATGTTGC
TGTTGTCGCAATGAAAGAAGAAGCAGCTGGTGAAGTTCCTGTTGCATTTGTGGTGAAATCGAAGGATTCGGAG
TTATCAGAAGATGATGTGAAGCAATTCGTGTCGAAACAGGTTGTGTTTTACAAGAGAATCAACAAAGTGTTCT
TCACTGAATCCATTCCTAAAGCTCCATCAGGGAAGATATTGAGGAAAGATCTGAGGGCAAAACTAGCAAATGG
ATTGTGA
Amino acid
Arabidopsis thaliana coumaroyl CoA ligase 1
SEQ ID NO: 2
MAPQEQAVSQVMEKQSNNNNSDVIFRSKLPDIYIPNHLSLHDYIFQNISEFATKPCLINGPTGHVYTYSDVHV
ISRQIAANFHKLGVNQNDVVMLLLPNCPEFVLSFLAASFRGATATAANPFFTPAEIAKQAKASNTKLIITEAR
YVDKIKPLQNDDGVVIVCIDDNESVPIPEGCLRFTELTQSTTEASEVIDSVEISPDDVVALPYSSGTTGLPKG
VMLTHKGLVTSVAQQVDGENPNLYFHSDDVILCVLPMFHIYALNSIMLCGLRVGAAILIMPKFEINLLWELIQ
RCKVTVAPMVPPIVLAIAKSSETEKYDLSSIRVVKSGAAPLGKELEDAVNAKFPNAKLGQGYGMTEAGPVLAM
SLGFAKEPFPVKSGACGTVVRNAEMKIVDPDTGDSLSRNQPGEICIRGHQIMKGYLNNPAATAETIDKDGWLH
TGDIGLIDDDDELFIVDRLKELIKYKGFQVAPAELEALLIGHPDITDVAVVAMKEEAAGEVPVAFVVKSKDSE
LSEDDVKQFVSKQVVFYKRINKVFFTESIPKAPSGKILRKDLRAKLANGL
Synthetic DNA
Codon optimized Vitis vinifera stilbene synthase opt1
SEQ ID NO: 3
ATGGCTTCTGTTGAGGAATTTAGGAATGCTCAACGTGCCAAGGGACCCGCCACTATTCTGGCTATAGGTACTG
CCACCCCAGATCATTGCGTATATCAATCGGATTACGCTGACTACTACTTCAAGGTTACCAAAAGTGAGCACAT
GACAGCCTTGAAGAAGAAGTTTAACCGTATATGCGATAAGTCAATGATCAAGAAAAGATACATTCACTTGACA
GAAGAAATGTTAGAGGAACATCCAAATATAGGCGCTTACATGGCTCCATCGTTAAACATCCGTCAGGAAATCA
TTACAGCTGAAGTACCCAAATTAGGTAAAGAGGCTGCATTGAAAGCCCTAAAAGAATGGGGCCAACCTAAATC
CAAAATTACTCATTTGGTATTCTGTACCACAAGCGGCGTTGAAATGCCTGGAGCTGACTATAAACTTGCCAAC
CTACTGGGCTTGGAACCTTCCGTCCGTAGGGTAATGCTTTACCACCAAGGTTGTTATGCTGGTGGGACAGTCT
TGAGGACGGCTAAGGACTTAGCCGAAAATAATGCTGGGGCACGGGTTCTAGTTGTATGTTCGGAAATTACGGT
TGTAACTTTTCGTGGTCCATCAGAAGATGCATTAGATTCGTTGGTCGGTCAGGCATTATTTGGCGATGGCTCC
GCAGCAGTCATCGTCGGTTCGGATCCAGATATTAGTATAGAGCGCCCCTTGTTCCAACTCGTATCCGCAGCTC
AAACATTTATTCCAAACTCCGCGGGTGCGATTGCCGGGAACTTACGGGAAGTGGGTTTAACCTTTCACCTCTG
GCCAAATGTTCCTACCCTTATTTCCGAAAACGTTGAGAAATGCCTAACACAAGCTTTCGATCCTCTAGGAATC
TCGGATTGGAATAGCTTGTTCTGGATTGCCCATCCAGGTGGTCCTGCCATTCTTGATGCGGTTGAGGCTAAAT
TGAACCTAGACAAGAAGAAGTTGGAAGCCACAAGACATGTACTGTCAGAATATGGAAATATGAGTTCTGCCTG
TGTCTTATTCATACTCGACGAAATGAGAAAGAAGTCCTTAAAGGGCGAAAGAGCTACTACCGGCGAAGGACTA
GATTGGGGAGTTTTGTTTGGTTTCGGTCCTGGATTGACAATTGAAACAGTTGTTTTGCATAGTATTCCCATGG
TTACCAATTAA
Synthetic DNA
Codon optimized Vitis vinifera stilbene synthase opt2
SEQ ID NO: 4
ATGGCTAGCGTGGAGGAATTTAGGAATGCACAGAGAGCGAAAGGGCCTGCTACCATTTTAGCAATCGGTACTG
CGACTCCAGATCATTGTGTATACCAAAGTGATTATGCAGACTATTATTTCAAGGTCACCAAGTCTGAACACAT
GACCGCATTAAAGAAGAAGTTTAATAGAATATGCGATAAGAGCATGATCAAGAAACGTTATATTCACTTGACG
GAAGAAATGTTGGAAGAACATCCTAATATAGGTGCTTACATGGCACCCTCTTTGAATATCAGACAGGAAATAA
TTACGGCAGAAGTTCCCAAATTGGGAAAAGAGGCTGCCTTGAAGGCTTTAAAAGAATGGGGTCAGCCCAAATC
TAAAATTACCCACTTAGTATTTTGTACGACATCAGGCGTCGAAATGCCAGGTGCGGATTACAAATTAGCCAAT
TTGTTAGGTTTGGAACCGTCAGTTAGACGTGTTATGTTGTACCATCAAGGATGCTATGCCGGTGGGACGGTTC
TGAGAACAGCGAAAGATCTAGCTGAGAATAACGCAGGCGCAAGAGTATTGGTAGTCTGTTCCGAAATAACTGT
TGTCACTTTCAGAGGCCCAAGTGAGGACGCGTTGGACTCATTAGTTGGTCAGGCACTGTTTGGCGATGGTTCT
GCCGCTGTAATTGTCGGTAGCGACCCTGATATAAGTATTGAAAGACCCCTGTTCCAATTGGTTTCAGCAGCAC
AAACTTTTATTCCTAATAGTGCTGGTGCTATCGCTGGTAATTTAAGAGAAGTTGGCTTAACATTTCATTTGTG
GCCTAATGTTCCAACCCTGATAAGCGAAAACGTAGAGAAATGTCTTACCCAAGCGTTCGACCCATTAGGAATT
AGTGATTGGAACTCTCTTTTCTGGATCGCACACCCAGGAGGCCCAGCTATATTAGACGCAGTTGAAGCTAAGT
TAAATTTAGATAAGAAGAAATTGGAGGCAACAAGACATGTGTTATCCGAGTACGGAAATATGTCATCAGCATG
TGTGTTGTTTATATTGGACGAGATGAGAAAGAAGAGTCTTAAGGGAGAGAGAGCTACCACAGGAGAGGGATTG
GATTGGGGTGTCTTATTTGGTTTTGGTCCAGGTCTAACAATTGAAACAGTAGTGTTACACTCTATTCCAATGG
TCACAAATTAA
Synthetic DNA
Codon optimized Vitis vinifera stilbene synthase opt3
SEQ ID NO: 5
ATGGCATCCGTGGAAGAATTTAGAAACGCACAGAGGGCAAAAGGTCCAGCAACCATACTAGCTATCGGCACAG
CTACCCCTGATCATTGCGTCTATCAGTCGGACTACGCTGATTATTATTTTAAGGTTACCAAATCAGAACACAT
GACCGCATTGAAGAAGAAGTTTAACAGAATATGTGACAAATCAATGATTAAGAAGCGCTATATTCATCTAACT
GAGGAGATGCTGGAGGAACATCCAAATATTGGTGCGTACATGGCACCATCCCTAAACATTCGCCAAGAGATTA
TTACGGCTGAAGTTCCCAAGTTAGGCAAGGAAGCAGCTCTGAAGGCATTAAAGGAGTGGGGCCAGCCTAAGAG
CAAAATCACTCATCTTGTATTTTGTACGACCTCTGGTGTGGAAATGCCTGGAGCTGACTATAAATTAGCGAAC
TTGTTGGGCCTAGAGCCAAGTGTTAGAAGGGTGATGCTGTATCATCAGGGTTGTTATGCAGGTGGTACTGTCT
TGAGGACAGCCAAGGATCTGGCTGAAAATAATGCTGGCGCCAGAGTACTCGTAGTATGCAGTGAGATCACCGT
CGTCACATTTAGGGGACCATCTGAAGATGCTTTGGATTCTCTCGTTGGCCAGGCTTTATTCGGCGATGGTTCC
GCTGCTGTGATAGTCGGCTCGGATCCTGACATATCCATCGAACGCCCCTTGTTTCAATTAGTTAGCGCAGCGC
AGACCTTTATACCTAACTCGGCCGGGGCAATAGCAGGTAATTTGCGTGAAGTCGGATTGACTTTTCATTTGTG
GCCTAACGTCCCCACGTTGATTTCAGAAAATGTCGAAAAGTGTTTAACGCAAGCATTCGATCCTCTAGGTATA
TCTGATTGGAATAGCCTCTTCTGGATTGCACATCCTGGCGGGCCTGCTATTCTGGACGCGGTCGAGGCTAAGT
TAAATTTGGATAAGAAGAAGCTGGAAGCCACCAGACATGTCCTGTCTGAGTACGGGAATATGTCAAGTGCATG
TGTGCTCTTTATACTGGACGAGATGAGGAAGAAATCGTTAAAGGGTGAGAGAGCTACTACGGGTGAAGGATTA
GATTGGGGCGTATTATTCGGCTTCGGTCCGGGGCTCACTATCGAAACAGTAGTCCTGCATAGTATCCCCATGG
TCACCAATTGA
Synthetic DNA
Codon optimized Vitis vinifera stilbene synthase opt4
SEQ ID NO: 6
ATGGCCTCAGTAGAAGAGTTTCGTAATGCTCAAAGAGCCAAGGGCCCAGCTACAATTTTAGCTATAGGCACCG
CTACGCCAGATCATTGTGTTTACCAATCCGATTACGCAGATTACTATTTCAAGGTCACAAAGAGCGAACACAT
GACTGCCTTAAAGAAGAAATTTAACCGTATCTGTGACAAATCTATGATCAAGAAGCGTTACATACATTTGACT
GAAGAGATGTTAGAGGAGCACCCTAACATTGGTGCCTACATGGCACCGTCGTTAAATATCCGTCAAGAAATTA
TTACAGCTGAGGTCCCAAAGTTAGGTAAGGAAGCTGCTCTTAAAGCCTTGAAGGAATGGGGTCAACCTAAGAG
TAAAATTACACATTTGGTCTTTTGTACCACTTCCGGCGTTGAAATGCCTGGCGCCGATTACAAGTTAGCTAAC
CTATTAGGTCTGGAACCAAGCGTTCGTCGCGTAATGTTATACCATCAGGGATGTTATGCAGGTGGTACTGTAT
TAAGGACCGCAAAAGACTTGGCAGAAAATAACGCGGGCGCCAGAGTATTGGTCGTGTGTAGCGAAATTACGGT
TGTAACATTCAGGGGTCCATCAGAGGACGCACTGGACAGTCTCGTAGGGCAAGCACTATTTGGTGATGGAAGC
GCTGCGGTCATTGTTGGTAGCGACCCAGACATATCAATTGAAAGACCTCTTTTCCAACTTGTCTCTGCTGCCC
AAACTTTTATTCCGAATAGCGCCGGGGCTATCGCGGGTAATCTTAGAGAAGTGGGACTGACGTTTCATTTATG
GCCAAATGTGCCCACACTTATAAGCGAAAATGTCGAAAAATGTCTTACGCAGGCATTCGATCCTCTTGGTATA
TCGGATTGGAACTCTCTCTTTTGGATCGCCCATCCAGGTGGTCCTGCAATTCTGGATGCTGTAGAAGCAAAAC
TAAACCTGGACAAGAAGAAACTGGAAGCTACAAGACATGTCTTGTCGGAATACGGGAACATGAGTTCGGCATG
TGTACTTTTTATTTTAGATGAGATGCGTAAAAAGTCTCTGAAAGGTGAGCGTGCAACAACCGGTGAAGGTTTG
GACTGGGGTGTCTTGTTCGGATTCGGTCCCGGCTTAACCATCGAAACTGTAGTTCTACATTCTATTCCAATGG
TTACTAATTAA
Synthetic DNA
Codon optimized Vitis vinifera stilbene synthase opt5
SEQ ID NO: 7
ATGGCTTCAGTCGAGGAGTTTAGAAATGCTCAGAGGGCCAAGGGTCCTGCCACAATATTAGCTATAGGTACTG
CCACCCCAGATCACTGTGTCTATCAAAGTGACTATGCTGACTATTATTTTAAAGTCACAAAAAGTGAGCACAT
GACTGCATTGAAAAAGAAATTCAATAGGATATGTGATAAATCAATGATCAAAAAGAGATACATTCATCTAACT
GAGGAAATGTTAGAAGAGCATCCAAATATTGGTGCATATATGGCTCCATCCTTAAATATCAGACAGGAAATAA
TAACCGCTGAGGTGCCTAAACTGGGTAAAGAAGCTGCATTAAAAGCATTAAAAGAATGGGGTCAGCCTAAATC
AAAGATTACGCATCTAGTATTTTGCACAACGTCTGGTGTCGAAATGCCTGGAGCCGATTACAAACTAGCAAAT
TTACTAGGTCTTGAACCTTCTGTCCGTCGAGTAATGTTATACCACCAAGGTTGCTACGCAGGCGGAACCGTTC
TAAGGACTGCCAAGGACTTGGCAGAAAATAACGCTGGTGCAAGGGTTTTAGTGGTTTGTTCTGAAATCACTGT
AGTCACATTTAGGGGTCCCTCTGAAGATGCATTAGACTCTTTAGTTGGGCAAGCACTGTTCGGGGATGGGTCT
GCGGCCGTTATAGTAGGTTCAGATCCTGACATTTCTATCGAAAGGCCTCTGTTTCAACTGGTATCTGCTGCCC
AAACTTTTATTCCTAACAGCGCTGGTGCAATCGCCGGGAACCTCCGAGAAGTAGGTCTTACATTTCATCTATG
GCCTAATGTCCCTACTTTGATTTCCGAGAATGTAGAGAAATGCCTGACTCAGGCCTTTGATCCTTTGGGCATA
TCTGATTGGAACTCACTATTTTGGATTGCACACCCCGGAGGTCCCGCAATTTTGGATGCCGTGGAGGCTAAAT
TAAATTTAGATAAGAAGAAACTCGAAGCAACTAGACATGTATTATCAGAGTACGGCAATATGTCTAGTGCTTG
TGTTTTATTTATTTTAGACGAAATGCGTAAAAAGTCTTTAAAGGGAGAGAGGGCTACTACAGGAGAAGGATTA
GATTGGGGTGTTTTGTTTGGTTTCGGACCCGGTTTAACGATCGAAACAGTTGTTCTGCATAGTATCCCTATGG
TGACCAATTGA
Synthetic DNA
Codon optimized Vitis vinifera stilbene synthase opt6
SEQ ID NO: 8
ATGGCATCGGTAGAAGAGTTCAGAAATGCACAGAGGGCTAAAGGCCCTGCCACAATCCTAGCAATTGGTACTG
CAACTCCCGATCATTGCGTTTATCAAAGTGATTATGCCGACTATTATTTTAAAGTTACGAAATCAGAACACAT
GACTGCTCTTAAAAAGAAATTCAACAGAATATGTGACAAGAGTATGATTAAAAAGAGATACATTCACTTGACA
GAAGAGATGTTGGAGGAGCATCCTAATATCGGCGCTTACATGGCACCTTCATTGAACATTCGTCAAGAAATAA
TTACTGCCGAGGTTCCTAAACTCGGCAAAGAAGCAGCACTTAAGGCACTTAAGGAATGGGGTCAGCCAAAGTC
AAAGATCACACATTTGGTCTTTTGTACAACCTCTGGAGTTGAGATGCCAGGCGCTGATTATAAATTGGCTAAT
CTTTTAGGATTAGAGCCAAGTGTTAGGCGGGTGATGCTATATCACCAAGGTTGTTATGCAGGTGGTACTGTTT
TGAGGACAGCCAAGGATCTGGCCGAAAATAATGCTGGGGCCAGAGTCCTGGTTGTTTGCTCCGAGATAACTGT
TGTTACATTTCGCGGGCCTTCAGAAGATGCACTGGATTCTCTTGTGGGACAGGCGCTGTTTGGTGATGGGTCC
GCTGCCGTGATCGTAGGCTCTGATCCAGATATATCAATTGAGAGGCCTTTATTTCAGTTGGTGTCTGCCGCTC
AGACATTCATCCCTAATTCCGCGGGAGCGATAGCTGGTAATCTAAGAGAGGTTGGCTTGACATTTCACTTATG
GCCTAATGTGCCAACATTGATCTCTGAGAACGTCGAAAAGTGCCTAACCCAAGCATTTGACCCATTAGGAATT
AGCGACTGGAATAGTTTATTTTGGATAGCACACCCTGGAGGTCCGGCTATATTGGATGCTGTGGAAGCAAAGC
TAAATCTGGATAAGAAGAAGCTAGAAGCAACAAGACACGTACTATCTGAATACGGAAATATGAGCAGTGCTTG
TGTTCTATTTATTCTTGATGAGATGCGTAAAAAGAGTTTAAAtGGAGAAAGAGCCACCACAGGTGAAGGGCTA
GACTGGGGCGTTTTATTTGGCTTCGGTCCAGGTCTGACAATCGAAACGGTCGTCTTACACTCAATTCCAATGG
TTACAAATTGA
Amino acid
Vitis vinifera stilbene synthase
SEQ ID NO: 9
MASVEEFRNAQRAKGPATILAIGTATPDHCVYQSDYADYYFKVTKSEHMTALKKKFNRICDKSMIKKRYIHLT
EEMLEEHPNIGAYMAPSLNIRQEIITAEVPKLGKEAALKALKEWGQPKSKITHLVFCTTSGVEMPGADYKLAN
LLGLEPSVRRVMLYHQGCYAGGTVLRTAKDLAENNAGARVLVVCSEITVVTFRGPSEDALDSLVGQALFGDGS
AAVIVGSDPDISIERPLFQLVSAAQTFIPNSAGAIAGNLREVGLTFHLWPNVPTLISENVEKCLTQAFDPLGI
SDWNSLFWIAHPGGPAILDAVEAKLNLDKKKLEATRHVLSEYGNMSSACVLFILDEMRKKSLKGERATTGEGL
DWGVLFGFGPGLTIETVVLHSIPMVTN
DNA
Modified Saccharomyces cerevisiae acetyl CoA carboxylase 1
SEQ ID NO: 10
ATGAGCGAAGAAAGCTTATTCGAGTCTTCTCCACAGAAGATGGAGTACGAAATTACAAACTACTCAGAAAGAC
ATACAGAACTTCCAGGTCATTTCATTGGCCTCAATACAGTAGATAAACTAGAGGAGTCCCCGTTAAGGGACTT
TGTTAAGAGTCACGGTGGTCACACGGTCATATCCAAGATCCTGATAGCAAATAATGGTATTGCCGCCGTGAAA
GAAATTAGATCCGTCAGAAAATGGGCATACGAGACGTTCGGCGATGACAGAACCGTCCAATTCGTCGCCATGG
CCACCCCAGAAGATCTGGAGGCCAACGCAGAATATATCCGTATGGCCGATCAATACATTGAAGTGCCAGGTGG
TACTAATAATAACAACTACGCTAACGTAGACTTGATCGTAGACATCGCCGAAAGAGCAGACGTAGACGCCGTA
TGGGCTGGCTGGGGTCACGCCTCCGAGAATCCACTATTGCCTGAAAAATTGTCCCAGTCTAAGAGGAAAGTCA
TCTTTATTGGGCCTCCAGGTAACGCCATGAGGTCTTTAGGTGATAAAATCTCCTCTACCATTGTCGCTCAAAG
TGCTAAAGTCCCATGTATTCCATGGTCTGGTACCGGTGTTGACACCGTTCACGTGGACGAGAAAACCGGTCTG
GTCTCTGTCGACGATGACATCTATCAAAAGGGTTGTTGTACCTCTCCTGAAGATGGTTTACAAAAGGCCAAGC
GTATTGGTTTTCCTGTCATGATTAAGGCATCCGAAGGTGGTGGTGGTAAAGGTATCAGACAAGTTGAACGTGA
AGAAGATTTCATCGCTTTATACCACCAGGCAGCCAACGAAATTCCAGGCTCCCCCATTTTCATCATGAAGTTG
GCCGGTAGAGCGCGTCACTTGGAAGTTCAACTGCTAGCAGATCAGTACGGTACAAATATTTCCTTGTTCGGTA
GAGACTGTTCCGTTCAGAGACGTCATCAAAAAATTATCGAAGAAGCACCAGTTACAATTGCCAAGGCTGAAAC
ATTTCACGAGATGGAAAAGGCTGCCGTCAGACTGGGGAAACTAGTCGGTTATGTCTCTGCCGGTACCGTGGAG
TATCTATATTCTCATGATGATGGAAAATTCTACTTTTTAGAATTGAACCCAAGATTACAAGTCGAGCATCCAA
CAACGGAAATGGTCTCCGGTGTTAACTTACCTGCAGCTCAATTACAAATCGCTATGGGTATCCCTATGCATAG
AATAAGTGACATTAGAACTTTATATGGTATGAATCCTCATTCTGCCTCAGAAATCGATTTCGAATTCAAAACT
CAAGATGCCACCAAGAAACAAAGAAGACCTATTCCAAAGGGTCATTGTACCGCTTGTCGTATCACATCAGAAG
ATCCAAACGATGGATTCAAGCCATCGGGTGGTACTTTGCATGAACTAAACTTCCGTTCTTCCTCTAATGTTTG
GGGTTACTTCTCCGTGGGTAACAATGGTAATATTCACTCCTTTTCGGACTCTCAGTTCGGCCATATTTTTGCT
TTTGGTGAAAATAGACAAGCTTCCAGGAAACACATGGTTGTTGCCCTGAAGGAATTGTCCATTAGGGGTGATT
TCAGAACTACTGTGGAATACTTGATCAAACTTTTGGAAACTGAAGATTTCGAGGATAACACTATTACCACCGG
TTGGTTGGACGATTTGATTACTCATAAAATGACCGCTGAAAAGCCTGATCCAACTCTTGCCGTCATTTGCGGT
GCCGCTACAAAGGCTTTCTTAGCATCTGAAGAAGCCCGCCACAAGTATATCGAATCCTTACAAAAGGGACAAG
TTCTATCTAAAGACCTACTGCAAACTATGTTCCCTGTAGATTTTATCCATGAGGGTAAAAGATACAAGTTCAC
CGTAGCTAAATCCGGTAATGACCGTTACACATTATTTATCAATGGTTCTAAATGTGATATCATACTGCGTCAA
CTAGCTGATGGTGGTCTTTTGATTGCCATAGGCGGTAAATCGCATACCATCTATTGGAAAGAAGAAGTTGCTG
CTACAAGATTATCCGTTGACTCTATGACTACTTTGTTGGAAGTTGAAAACGATCCAACCCAGTTGCGTACTCC
ATCCCCTGGTAAATTGGTTAAATTCTTGGTGGAAAATGGTGAACACATTATCAAGGGCCAACCATATGCAGAA
ATTGAAGTTATGAAAATGCAAATGCCTTTGGTTTCTCAAGAAAATGGTATCGTCCAGTTATTAAAGCAACCTG
GTTCTACCATTGTTGCAGGTGATATCATGGCTATTATGACTCTTGACGATCCATCCAAGGTCAAGCACGCTCT
ACCATTTGAAGGTATGCTGCCAGATTTTGGTTCTCCAGTTATCGAAGGAACCAAACCTGCCTATAAATTCAAG
TCATTAGTGTCTACTTTGGAAAACATTTTGAAGGGTTATGACAACCAAGTTATTATGAACGCTTCCTTGCAAC
AATTGATAGAGGTTTTGAGAAATCCAAAACTGCCTTACTCAGAATGGAAACTACACATCTCTGCTTTACATTC
AAGATTGCCTGCTAAGCTAGATGAACAAATGGAAGAGTTAGTTGCACGTTCTTTGAGACGTGGTGCTGTTTTC
CCAGCTAGACAATTAAGTAAATTGATTGATATGGCCGTGAAGAATCCTGAATACAACCCCGACAAATTGCTGG
GCGCCGTCGTGGAACCATTGGCGGATATTGCTCATAAGTACTCTAACGGGTTAGAAGCCCATGAACATTCTAT
ATTTGTCCATTTCTTGGAAGAATATTACGAAGTTGAAAAGTTATTCAATGGTCCAAATGTTCGTGAGGAAAAT
ATCATTCTGAAATTGCGTGATGAAAACCCTAAAGATCTAGATAAAGTTGCGCTAACTGTTTTGTCTCATTCGA
AAGTTTCAGCGAAGAATAACCTGATCCTAGCTATCTTGAAACATTATCAACCATTGTGCAAGTTATCTTCTAA
AGTTTCTGCCATTTTCTCTACTCCTCTACAACATATTGTTGAACTAGAATCTAAGGCTACCGCTAAGGTCGCT
CTACAAGCAAGAGAAATTTTGATTCAAGGCGCTTTACCTTCGGTCAAGGAAAGAACTGAACAAATTGAACATA
TCTTAAAATCCTCTGTTGTGAAGGTTGCCTATGGCTCATCCAATCCAAAGCGCTCTGAACCAGATTTGAATAT
CTTGAAGGACTTGATCGATTCTAATTACGTTGTGTTCGATGTTTTACTTCAATTCCTAACCCATCAAGACCCA
GTTGTGACTGCTGCAGCTGCTCAAGTCTATATTCGTCGTGCTTATCGTGCTTACACCATAGGAGATATTAGAG
TTCACGAAGGTGTCACAGTTCCAATTGTTGAATGGAAATTCCAACTACCTTCAGCTGCGTTCTCCACCTTTCC
AACTGTTAAATCTAAAATGGGTATGAACAGGGCTGTTGCTGTTTCAGATTTGTCATATGTTGCAAACAGTCAG
TCATCTCCGTTAAGAGAAGGTATTTTGATGGCTGTGGATCATTTAGATGATGTTGATGAAATTTTGTCACAAA
GTTTGGAAGTTATTCCTCGTCACCAATCTTCTTCTAACGGACCTGCTCCTGATCGTTCTGGTAGCTCCGCATC
GTTGAGTAATGTTGCTAATGTTTGTGTTGCTTCTACAGAAGGTTTCGAATCTGAAGAGGAAATTTTGGTAAGG
TTGAGAGAAATTTTGGATTTGAATAAGCAGGAATTAATCAATGCTTCTATCCGTCGTATCACATTTATGTTCG
GTTTTAAAGATGGGTCTTATCCAAAGTATTATACTTTTAACGGTCCAAATTATAACGAAAATGAAACAATTCG
TCACATTGAGCCGGCTTTGGCCTTCCAACTGGAATTAGGAAGATTGTCCAACTTCAACATTAAACCAATTTTC
ACTGATAATAGAAACATCCATGTCTACGAAGCTGTTAGTAAGACTTCTCCATTGGATAAGAGATTCTTTACAA
GAGGTATTATTAGAACGGGTCATATCCGTGATGACATTTCTATTCAAGAATATCTGACTTCTGAAGCTAACAG
ATTGATGAGTGATATATTGGATAATTTAGAAGTCACCGACACTTCAAATTCTGATTTGAATCATATCTTCATC
AACTTCATTGCGGTGTTTGATATCTCTCCAGAAGATGTCGAAGCCGCCTTCGGTGGTTTCTTAGAAAGATTTG
GTAAGAGATTGTTGAGATTGCGTGTTTCTTCTGCCGAAATTAGAATCATCATCAAAGATCCTCAAACAGGTGC
CCCAGTACCATTGCGTGCCTTGATCAATAACGTTTCTGGTTATGTTATCAAAACAGAAATGTACACCGAAGTC
AAGAACGCAAAAGGTGAATGGGTATTTAAGTCTTTGGGTAAACCTGGATCCATGCATTTAAGACCTATTGCTA
CTCCTTACCCTGTTAAGGAATGGTTGCAACCAAAACGTTATAAGGCACACTTGATGGGTACCACATATGTCTA
TGACTTCCCAGAATTATTCCGCCAAGCATCGTCATCCCAATGGAAAAATTTCTCTGCAGATGTTAAGTTAACA
GATGATTTCTTTATTTCCAACGAGTTGATTGAAGATGAAAACGGCGAATTAACTGAGGTGGAAAGAGAACCTG
GTGCCAACGCTATTGGTATGGTTGCCTTTAAGATTACTGTAAAGACTCCTGAATATCCAAGAGGCCGTCAATT
TGTTGTTGTTGCTAACGATATCACATTCAAGATCGGTTCCTTTGGTCCACAAGAAGACGAATTCTTCAATAAG
GTTACTGAATATGCTAGAAAGCGTGGTATCCCAAGAATTTACTTGGCTGCAAACTCAGGTGCCAGAATTGGTA
TGGCTGAAGAGATTGTTCCACTATTTCAAGTTGCATGGAATGATGCTGCCAATCCGGACAAGGGCTTCCAATA
CTTATACTTAACAAGTGAAGGTATGGAAACTTTAAAGAAATTTGACAAAGAAAATTCTGTTCTCACTGAACGT
ACTGTTATAAACGGTGAAGAAAGATTTGTCATCAAGACAATTATTGGTTCTGAAGATGGGTTAGGTGTCGAAT
GTCTACGTGGATCTGGTTTAATTGCTGGTGCAACGTCAAGGGCTTACCACGATATCTTCACTATCACCTTAGT
CACTTGTAGATCCGTCGGTATCGGTGCTTATTTGGTTCGTTTGGGTCAAAGAGCTATTCAGGTCGAAGGCCAG
CCAATTATTTTAACTGGTGCTCCTGCAATCAACAAAATGCTGGGTAGAGAAGTTTATACTTCTAACTTACAAT
TGGGTGGTACTCAAATCATGTATAACAACGGTGTTTCACATTTGACTGCTGTTGACGATTTAGCTGGTGTAGA
GAAGATTGTTGAATGGATGTCTTATGTTCCAGCCAAGCGTAATATGCCAGTTCCTATCTTGGAAACTAAAGAC
ACATGGGATAGACCAGTTGATTTCACTCCAACTAATGATGAAACTTACGATGTAAGATGGATGATTGAAGGTC
GTGAGACTGAAAGTGGATTTGAATATGGTTTGTTTGATAAAGGGTCTTTCTTTGAAACTTTGTCAGGATGGGC
CAAAGGTGTTGTCGTTGGTAGAGCCCGTCTTGGTGGTATTCCACTGGGTGTTATTGGTGTTGAAACAAGAACT
GTCGAGAACTTGATTCCTGCTGATCCAGCTAATCCAAATAGTGCTGAAACATTAATTCAAGAACCTGGTCAAG
TTTGGCATCCAAACTCCGCCTTCAAGACTGCTCAAGCTATCAATGACTTTAACAACGGTGAACAATTGCCAAT
GATGATTTTGGCCAACTGGAGAGGTTTCTCTGGTGGTCAACGTGATATGTTCAACGAAGTCTTGAAGTATGGT
TCGTTTATTGTTGACGCATTGGTGGATTACAAACAACCAATTATTATCTATATCCCACCTACCGGTGAACTAA
GAGGTGGTTCATGGGTTGTTGTCGATCCAACTATCAACGCTGACCAAATGGAAATGTATGCCGACGTCAACGC
TAGAGCTGGTGTTTTGGAACCACAAGGTATGGTTGGTATCAAGTTCCGTAGAGAAAAATTGCTGGACACCATG
AACAGATTGGATGACAAGTACAGAGAATTGAGATCTCAATTATCCAACAAGAGTTTGGCTCCAGAAGTACATC
AGCAAATATCCAAGCAATTAGCTGATCGTGAGAGAGAACTATTGCCAATTTACGGACAAATCAGTCTTCAATT
TGCTGATTTGCACGATAGGTCTTCACGTATGGTGGCCAAGGGTGTTATTTCTAAGGAACTGGAATGGACCGAG
GCACGTCGTTTCTTCTTCTGGAGATTGAGAAGAAGATTGAACGAAGAATATTTGATTAAAAGGTTGAGCCATC
AGGTAGGCGAAGCATCAAGATTAGAAAAGATCGCAAGAATTAGATCGTGGTACCCTGCTTCAGTGGACCATGA
AGATGATAGGCAAGTCGCAACATGGATTGAAGAAAACTACAAAACTTTGGACGATAAACTAAAGGGTTTGAAA
TTAGAGTCATTCGCTCAAGACTTAGCTAAAAAGATCAGAAGCGACCATGACAATGCTATTGATGGATTATCTG
AAGTTATCAAGATGTTATCTACCGATGATAAAGAAAAATTGTTGAAGACTTTGAAATAA
Amino acid
Modified Saccharomyces cerevisiae acetyl CoA carboxylase 1
SEQ ID NO: 11
MSEESLFESSPQKMEYEITNYSERHTELPGHFIGLNTVDKLEESPLRDFVKSHGGHTVISKILIANNGIAAVK
EIRSVRKWAYETFGDDRTVQFVAMATPEDLEANAEYIRMADQYIEVPGGTNNNNYANVDLIVDIAERADVDAV
WAGWGHASENPLLPEKLSQSKRKVIFIGPPGNAMRSLGDKISSTIVAQSAKVPCIPWSGTGVDTVHVDEKTGL
VSVDDDIYQKGCCTSPEDGLQKAKRIGFPVMIKASEGGGGKGIRQVEREEDFIALYHQAANEIPGSPIFIMKL
AGRARHLEVQLLADQYGTNISLFGRDCSVQRRHQKIIEEAPVTIAKAETFHEMEKAAVRLGKLVGYVSAGTVE
YLYSHDDGKFYFLELNPRLQVEHPTTEMVSGVNLPAAQLQIAMGIPMHRISDIRTLYGMNPHSASEIDFEFKT
QDATKKQRRPIPKGHCTACRITSEDPNDGFKPSGGTLHELNFRSSSNVWGYFSVGNNGNIHSFSDSQFGHIFA
FGENRQASRKHMVVALKELSIRGDFRTTVEYLIKLLETEDFEDNTITTGWLDDLITHKMTAEKPDPTLAVICG
AATKAFLASEEARHKYIESLQKGQVLSKDLLQTMFPVDFIHEGKRYKFTVAKSGNDRYTLFINGSKCDIILRQ
LADGGLLIAIGGKSHTIYWKEEVAATRLSVDSMTTLLEVENDPTQLRTPSPGKLVKFLVENGEHIIKGQPYAE
IEVMKMQMPLVSQENGIVQLLKQPGSTIVAGDIMAIMTLDDPSKVKHALPFEGMLPDFGSPVIEGTKPAYKFK
SLVSTLENILKGYDNQVIMNASLQQLIEVLRNPKLPYSEWKLHISALHSRLPAKLDEQMEELVARSLRRGAVF
PARQLSKLIDMAVKNPEYNPDKLLGAVVEPLADIAHKYSNGLEAHEHSIFVHFLEEYYEVEKLFNGPNVREEN
IILKLRDENPKDLDKVALTVLSHSKVSAKNNLILAILKHYQPLCKLSSKVSAIFSTPLQHIVELESKATAKVA
LQAREILIQGALPSVKERTEQIEHILKSSVVKVAYGSSNPKRSEPDLNILKDLIDSNYVVFDVLLQFLTHQDP
VVTAAAAQVYIRRAYRAYTIGDIRVHEGVTVPIVEWKFQLPSAAFSTFPTVKSKMGMNRAVAVSDLSYVANSQ
SSPLREGILMAVDHLDDVDEILSQSLEVIPRHQSSSNGPAPDRSGSSASLSNVANVCVASTEGFESEEEILVR
LREILDLNKQELINASIRRITFMFGFKDGSYPKYYTFNGPNYNENETIRHIEPALAFQLELGRLSNFNIKPIF
TDNRNIHVYEAVSKTSPLDKRFFTRGIIRTGHIRDDISIQEYLTSEANRLMSDILDNLEVTDTSNSDLNHIFI
NFIAVFDISPEDVEAAFGGFLERFGKRLLRLRVSSAEIRIIIKDPQTGAPVPLRALINNVSGYVIKTEMYTEV
KNAKGEWVFKSLGKPGSMHLRPIATPYPVKEWLQPKRYKAHLMGTTYVYDFPELFRQASSSQWKNFSADVKLT
DDFFISNELIEDENGELTEVEREPGANAIGMVAFKITVKTPEYPRGRQFVVVANDITFKIGSFGPQEDEFFNK
VTEYARKRGIPRIYLAANSGARIGMAEEIVPLFQVAWNDAANPDKGFQYLYLTSEGMETLKKFDKENSVLTER
TVINGEERFVIKTIIGSEDGLGVECLRGSGLIAGATSRAYHDIFTITLVTCRSVGIGAYLVRLGQRAIQVEGQ
PIILTGAPAINKMLGREVYTSNLQLGGTQIMYNNGVSHLTAVDDLAGVEKIVEWMSYVPAKRNMPVPILETKD
TWDRPVDFTPTNDETYDVRWMIEGRETESGFEYGLFDKGSFFETLSGWAKGVVVGRARLGGIPLGVIGVETRT
VENLIPADPANPNSAETLIQEPGQVWHPNSAFKTAQAINDFNNGEQLPMMILANWRGFSGGQRDMFNEVLKYG
SFIVDALVDYKQPIIIYIPPTGELRGGSWVVVDPTINADQMEMYADVNARAGVLEPQGMVGIKFRREKLLDTM
NRLDDKYRELRSQLSNKSLAPEVHQQ1SKQLADRERELLPIYGQISLQFADLHDRSSRMVAKGVISKELEWTE
ARRFFFWRLRRRLNEEYLIKRLSHQVGEASRLEKIARIRSWYPASVDHEDDRQVATWIEENYKTLDDKLKGLK
LESFAQDLAKKIRSDHDNAIDGLSEVIKMLSTDDKEKLLKTLK
Synthetic DNA
Codon optimized Arabidopsis thaliana coumaroyl CoA ligase 2
SEQ ID NO: 12
ATGACTACGCAGGATGTTATTGTCAATGATCAAAATGACCAAAAGCAATGTTCGAATGATGTTATCTTTCGTA
GTAGACTCCCTGATATATACATACCTAACCATCTACCATTGCATGATTACATATTTGAAAATATATCGGAATT
TGCTGCTAAGCCATGCCTAATCAATGGTCCAACAGGTGAAGTGTATACCTATGCTGATGTTCATGTTACTTCC
AGGAAGCTCGCTGCTGGTTTGCACAACTTGGGCGTTAAACAGCATGACGTCGTTATGATATTGCTGCCAAATA
GCCCAGAAGTGGTACTTACTTTCTTGGCCGCCTCGTTTATTGGCGCCATTACGACATCCGCAAATCCCTTCTT
CACGCCCGCTGAAATTTCTAAACAAGCTAAAGCATCTGCTGCTAAATTAATCGTCACACAAAGTAGATATGTT
GATAAGATTAAGAACTTACAAAACGATGGGGTCTTAATTGTCACAACCGATTCTGATGCTATCCCTGAAAATT
GTCTGAGATTCTCTGAGTTAACTCAATCCGAAGAGCCTAGAGTAGACAGTATACCTGAGAAGATCTCTCCAGA
AGATGTGGTGGCTTTGCCATTTTCCTCAGGTACTACCGGTCTGCCAAAGGGTGTGATGTTGACTCACAAGGGT
TTGGTGACGTCAGTAGCTCAGCAAGTAGATGGGGAGAACCCTAATCTGTATTTCAATAGAGATGACGTCATTT
TGTGCGTATTACCTATGTTCCATATTTATGCATTAAACTCGATTATGCTATGCTCTCTGCGAGTTGGAGCAAC
TATATTAATCATGCCAAAGTTTGAGATAACTCTCTTGTTAGAACAAATTCAGAGGTGCAAGGTCACTGTTGCT
ATGGTAGTACCACCAATAGTCCTGGCAATCGCAAAGAGTCCTGAAACCGAGAAGTATGATTTAAGTAGTGTGC
GGATGGTTAAATCAGGCGCTGCCCCTCTAGGTAAAGAATTAGAAGATGCCATTTCCGCTAAATTTCCGAATGC
AAAATTAGGCCAAGGATATGGCATGACGGAAGCTGGTCCAGTTCTAGCAATGTCTTTGGGGTTTGCTAAAGAG
CCTTTTCCCGTAAAGAGCGGTGCCTGTGGCACTGTTGTGCGTAATGCTGAGATGAAAATACTGGATCCAGACA
CGGGCGATTCACTACCACGCAATAAACCAGGCGAGATATGTATAAGGGGAAACCAGATTATGAAGGGGTATTT
GAACGATCCCCTGGCCACCGCCTCAACTATCGATAAGGACGGATGGTTACACACTGGTGACGTTGGGTTTATT
GACGATGATGATGAATTATTCATCGTTGACAGATTAAAGGAATTGATCAAATACAAAGGTTTTCAAGTAGCTC
CAGCAGAACTCGAAAGCCTTTTGATTGGACATCCAGAGATAAATGACGTCGCAGTGGTCGCTATGAAAGAAGA
GGATGCTGGTGAAGTTCCCGTTGCATTTGTAGTTAGATCGAAGGATTCCAACATTAGCGAGGACGAAATTAAA
CAATTTGTAAGCAAACAGGTTGTCTTTTATAAAAGAATCAATAAAGTTTTCTTCACTGACTCAATTCCAAAGG
CCCCTTCTGGTAAAATCCTGCGTAAGGACTTGAGGGCACGATTGGCTAATGGCCTCATGAATTGA
Amino acid
Arabidopsis thaliana coumaroyl CoA ligase 2
SEQ ID NO: 13
MTTQDVIVNDQNDQKQCSNDVIFRSRLPDIYIPNHLPLHDYIFENISEFAAKPCLINGPTGEVYTYADVHVTS
RKLAAGLHNLGVKQHDVVMILLPNSPEVVLTFLAASFIGAITTSANPFFTPAEISKQAKASAAKLIVTQSRYV
DKIKNLQNDGVLIVTTDSDAIPENCLRFSELTQSEEPRVDSIPEKISPEDVVALPFSSGTTGLPKGVMLTHKG
LVTSVAQQVDGENPNLYFNRDDVILCVLPMFHIYALNSIMLCSLRVGATILIMPKFEITLLLEQIQRCKVTVA
MVVPPIVLAIAKSPETEKYDLSSVRMVKSGAAPLGKELEDAISAKFPNAKLGQGYGMTEAGPVLAMSLGFAKE
PFPVKSGACGTVVRNAEMKILDPDTGDSLPRNKPGEICIRGNQIMKGYLNDPLATASTIDKDGWLHTGDVGFI
DDDDELFIVDRLKELIKYKGFQVAPAELESLLIGHPEINDVAVVAMKEEDAGEVPVAFVVRSKDSNISEDEIK
QFVSKQVVFYKRINKVFFTDSIPKAPSGKILRKDLRARLANGLMN
Synthetic DNA
Plasmid sequence
SEQ ID NO: 14
GACGAAAGGGCCTCGTGATACGCCTATTTTTATAGGTTAATGTCATGATAATAATGGTTTCTTAGTATGATCC
AATATCAAAGGAAATGATAGCATTGAAGGATGAGACTAATCCAATTGAGGAGTGGCAGCATATAGAACAGCTA
AAGGGTAGTGCTGAAGGAAGCATACGATACCCCGCATGGAATGGGATAATATCACAGGAGGTACTAGACTACC
TTTCATCCTACATAAATAGACGCATATAAGTACGCATTTAAGCATAAACACGCACTATGCCGTTCTTCTCATG
TATATATATATACAGGCAACACGCAGATATAGGTGCGACGTGAACAGTGAGCTGTATGTGCGCAGCTCGCGTT
GCATTTTCGGAAGCGCTCGTTTTCGGAAACGCTTTGAAGTTCCTATTCCGAAGTTCCTATTCTCTAGAAAGTA
TAGGAACTTCAGAGCGCTTTTGAAAACCAAAAGCGCTCTGAAGACGCACTTTCAAAAAACCAAAAACGCACCG
GACTGTAACGAGCTACTAAAATATTGCGAATACCGCTTCCACAAACATTGCTCAAAAGTATCTCTTTGCTATA
TATCTCTGTGCTATATCCCTATATAACCTACCCATCCACCTTTCGCTCCTTGAACTTGCATCTAAACTCGACC
TCTACATTTTTTATGTTTATCTCTAGTATTACTCTTTAGACAAAAAAATTGTAGTAAGAACTATTCATAGAGT
GAATCGAAAACAATACGAAAATGTAAACATTTCCTATACGTAGTATATAGAGACAAAATAGAAGAAACCGTTC
ATAATTTTCTGACCAATGAAGAATCATCAACGCTATCACTTTCTGTTCACAAAGTATGCGCAATCCACATCGG
TATAGAATATAATCGGGGATGCCTTTATCTTGAAAAAATGCACCCGCAGCTTCGCTAGTAATCAGTAAACGCG
GGAAGTGGAGTCAGGCTTTTTTTATGGAAGAGAAAATAGACACCAAAGTAGCCTTCTTCTAACCTTAACGGAC
CTACAGTGCAAAAAGTTATCAAGAGACTGCATTATAGAGCGCACAAAGGAGAAAAAAAGTAATCTAAGATGCT
TTGTTAGAAAAATAGCGCTCTCGGGATGCATTTTTGTAGAACAAAAAAGAAGTATAGATTCTTTGTTGGTAAA
ATAGCGCTCTCGCGTTGCATTTCTGTTCTGTAAAAATGCAGCTCAGATTCTTTGTTTGAAAAATTAGCGCTCT
CGCGTTGCATTTTTGTTTTACAAAAATGAAGCACAGATTCTTCGTTGGTAAAATAGCGCTTTCGCGTTGCATT
TCTGTTCTGTAAAAATGCAGCTCAGATTCTTTGTTTGAAAAATTAGCGCTCTCGCGTTGCATTTTTGTTCTAC
AAAATGAAGCACAGATGCTTCGTTCAGGTGGCACTTTTCGGGGAAATGTGCGCGGAACCCCTATTTGTTTATT
TTTCTAAATACATTCAAATATGTATCCGCTCATGAGACAATAACCCTGATAAATGCTTCAATAATATTGAAAA
AGGAAGAGTATGAGTATTCAACATTTCCGTGTCGCCCTTATTCCCTTTTTTGCGGCATTTTGCCTTCCTGTTT
TTGCTCACCCAGAAACGCTGGTGAAAGTAAAAGATGCTGAAGATCAGTTGGGTGCACGAGTGGGTTACATCGA
ACTGGATCTCAACAGCGGTAAGATCCTTGAGAGTTTTCGCCCCGAAGAACGTTTTCCAATGATGAGCACTTTT
AAAGTTCTGCTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGACAGTAAGAGA
ATTATGCAGTGCTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGAACCGGAGCTGA
ATGAAGCCATACCAAACGACGAGCGTGACACCACGATGCCTGTAGCAATGGCAACAACGTTGCGCAAACTATT
AACTGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGATTAAGCATTGGTAACTGTCA
GACCAAGTTTACTCATATATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGAGCGTCAGACCCCGTAGA
AAAGATCAAAGGATCTTCTTGAGATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCCGAAGGTAACTGGCTTCAGCAGAG
CGCAGATACCAAATACTGTTCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGAGCGCCCAATACGCA
AACCGCCTCTCCCCGCGCGTTGGCCGATTCATTAATGCAGCTGGCACGACAGGTTTCCCGACTGGAAAGCGGG
CAGTGAGCGCAACGCAATTAATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAGCTATGACCATGATTACGCCA
AGCGCGCAATTAACCCTCACTAAAGGGAACAAAAGCTGGAGCTCAGTTTATCATTATCAATACTCGCCATTTC
AAAGAATACGTAAATAATTAATAGTAGTGATTTTCCTAACTTTATTTAGTCAAAAAATTAGCCTTTTAATTCT
GCTGTAACCCGTACATGCCCAAAATAGGGGGCGGGTTACACAGAATATATAACATCGTAGGTGTCTGGGTGAA
CAGTTTATTCCTGGCATCCACTAAATATAATGGAGCCCGCTTTTTAAGCTGGCATCCAGAAAAAAAAAGAATC
CCAGCACCAAAATATTGTTTTCTTCACCAACCATCAGTTCATAGGTCCATTCTCTTAGCGCAACTACAGAGAA
CAGGGGCACAAACAGGCAAAAAACGGGCACAACCTCAATGGAGTGATGCAACCTGCCTGGAGTAAATGATGAC
ACAAGGCAATTGACCCACGCATGTATCTATCTCATTTTCTTACACCTTCTATTACCTTCTGCTCTCTCTGATT
TGGAAAAAGCTGAAAAAAAAGGTTGAAACCAGTTCCCTGAAATTATTCCCCTACTTGACTAATAAGTATATAA
AGACGGTAGGTATTGATTGTAATTCTGTAAATCTATTTCTTAAACTTCTTAAATTCTACTTTTATAGTTAGTC
TTTTTTTTAGTTTTAAAACACCAGAACTTAGTTTCGACGGATTCTAGAACTAGTTTAAAAAAAATGGCTTCTG
TTGAGGAATTTAGGAATGCTCAACGTGCCAAGGGACCCGCCACTATTCTGGCTATAGGTACTGCCACCCCAGA
TCATTGCGTATATCAATCGGATTACGCTGACTACTACTTCAAGGTTACCAAAAGTGAGCACATGACAGCCTTG
AAGAAGAAGTTTAACCGTATATGCGATAAGTCAATGATCAAGAAAAGATACATTCACTTGACAGAAGAAATGT
TAGAGGAACATCCAAATATAGGCGCTTACATGGCTCCATCGTTAAACATCCGTCAGGAAATCATTACAGCTGA
AGTACCCAAATTAGGTAAAGAGGCTGCATTGAAAGCCCTAAAAGAATGGGGCCAACCTAAATCCAAAATTACT
CATTTGGTATTCTGTACCACAAGCGGCGTTGAAATGCCTGGAGCTGACTATAAACTTGCCAACCTACTGGGCT
TGGAACCTTCCGTCCGTAGGGTAATGCTTTACCACCAAGGTTGTTATGCTGGTGGGACAGTCTTGAGGACGGC
TAAGGACTTAGCCGAAAATAATGCTGGGGCACGGGTTCTAGTTGTATGTTCGGAAATTACGGTTGTAACTTTT
CGTGGTCCATCAGAAGATGCATTAGATTCGTTGGTCGGTCAGGCATTATTTGGCGATGGCTCCGCAGCAGTCA
TCGTCGGTTCGGATCCAGATATTAGTATAGAGCGCCCCTTGTTCCAACTCGTATCCGCAGCTCAAACATTTAT
TCCAAACTCCGCGGGTGCGATTGCCGGGAACTTACGGGAAGTGGGTTTAACCTTTCACCTCTGGCCAAATGTT
CCTACCCTTATTTCCGAAAACGTTGAGAAATGCCTAACACAAGCTTTCGATCCTCTAGGAATCTCGGATTGGA
ATAGCTTGTTCTGGATTGCCCATCCAGGTGGTCCTGCCATTCTTGATGCGGTTGAGGCTAAATTGAACCTAGA
CAAGAAGAAGTTGGAAGCCACAAGACATGTACTGTCAGAATATGGAAATATGAGTTCTGCCTGTGTCTTATTC
ATACTCGACGAAATGAGAAAGAAGTCCTTAAAGGGCGAAAGAGCTACTACCGGCGAAGGACTAGATTGGGGAG
TTTTGTTTGGTTTCGGTCCTGGATTGACAATTGAAACAGTTGTTTTGCATAGTATTCCCATGGTTACCAATTA
ACTCGAGTCATGTAATTAGTTATGTCACGCTTACATTCACGCCCTCCCCCCACATCCGCTCTAACCGAAAAGG
AAGGAGTTAGACAACCTGAAGTCTAGGTCCCTATTTATTTTTTTATAGTTATGTTAGTATTAAGAACGTTATT
TATATTTCAAATTTTTCTTTTTTTTCTGTACAGACGCGTGTACGCATGTAACATTATACTGAAAACCTTGCTT
GAGAAGGTTTTGGGACGCTCGAAGGCTTTAATTTGCGGCCGGTACCCAATTCGCCCTATAGTGAGTCGTATTA
CGCGCGCTCACTGGCCGTCGTTTTACAACGTCGTGACTGGGAAAACCCTGGCGTTACCCAACTTAATCGCCTT
GCAGCACATCCCCCTTTCGCCAGCTGGCGTAATAGCGAAGAGGCCCGCACCGATCGCCCTTCCCAACAGTTGC
GCAGCCTGAATGGCGAATGGACGCGCCCTGTAGCGGCGCATTAAGCGCGGCGGGTGTGGTGGTTACGCGCAGC
GTGACCGCTACACTTGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCCTATCTCGGTCT
ATTCTTTTGATTTATAAGGGATTTTGCCGATTTCGGCCTATTGGTTAAAAAATGAGCTGATTTAACAAAAATT
TAACGCGAATTTTAACAAAATATTAACGCTTACAATTTCCTGATGCGGTATTTTCTCCTTACGCATCTGTGCG
GTATTTCACACCGCATAGGGTAATAACTGATATAATTAAATTGAAGCTCTAATTTGTGAGTTTAGTATACATG
CATTTACTTATAATACAGTTTTTTAGTTTTGCTGGCCGCATCTTCTCAAATATGCTTCCCAGCCTGCTTTTCT
GTAACGTTCACCCTCTACCTTAGCATCCCTTCCCTTTGCAAATAGTCCTCTTCCAACAATAATAATGTCAGAT
CCTGTAGAGACCACATCATCCACGGTTCTATACTGTTGACCCAATGCGTCTCCCTTGTCATCTAAACCCACAC
CGGGTGTCATAATCAACCAATCGTAACCTTCATCTCTTCCACCCATGTCTCTTTGAGCAATAAAGCCGATAAC
AAAATCTTTGTCGCTCTTCGCAATGTCAACAGTACCCTTAGTATATTCTCCAGTAGATAGGGAGCCCTTGCAT
GACAATTCTGCTAACATCAAAAGGCCTCTAGGTTCCTTTGTTACTTCTTCTGCCGCCTGCTTCAAACCGCTAA
CAATACCTGGGCCCACCACACCGTGTGCATTCGTAATGTCTGCCCATTCTGCTATTCTGTATACACCCGCAGA
GTACTGCAATTTGACTGTATTACCAATGTCAGCAAATTTTCTGTCTTCGAAGAGTAAAAAATTGTACTTGGCG
GATAATGCCTTTAGCGGCTTAACTGTGCCCTCCATGGAAAAATCAGTCAAGATATCCACATGTGTTTTTAGTA
AACAAATTTTGGGACCTAATGCTTCAACTAACTCCAGTAATTCCTTGGTGGTACGAACATCCAATGAAGCACA
CAAGTTTGTTTGCTTTTCGTGCATGATATTAAATAGCTTGGCAGCAACAGGACTAGGATGAGTAGCAGCACGT
TCCTTATATGTAGCTTTCGACATGATTTATCTTCGTTTCCTGCAGGTTTTTGTTCTGTGCAGTTGGGTTAAGA
ATACTGGGCAATTTCATGTTTCTTCAACACTACATATGCGTATATATACCAATCTAAGTCTGTGCTCCTTCCT
TCGTTCTTCCTTCTGTTCGGAGATTACCGAATCAAAAAAATTTCAAGGAAACCGAAATCAAAAAAAAGAATAA
AAAAAAAATGATGAATTGAAAAGGTGGTATGGTGCACTCTCAGTACAATCTGCTCTGATGCCGCATAGTTAAG
CCAGCCCCGACACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCGCGA
Salmonella enterica acetyl CoA synthetase, feedback inhibition
resistant mutant Synthetic DNA
SEQ ID NO: 15
CTATGATGGCATTGCAATGGCTTGCTTCTCTTCGAGCGGTTTTTCCACAACGCCAGGATCTGCGAGAGTGGAC
GTGTCACCTAAATTACTTGTATCTCCGGCGGCGATTTTTCGCAGGATTCTCCTCATAATTTTCCCACTCCTTG
TTTTTGGTAGAGAGTCTGTCCAATGTAAAACATCTGGAGTAGCCAAAGGCCCAATCTCTTTTCGAACCCAGTT
CCTTACCTCCGCATACAATTCTGGAGAAGGCTCTTCACCATGATTGAGTGTCACATAAGCATATATAGCTTGC
CCTTTGATAGCATGAGGGATGCCCACAACAGCCGCTTCAGCTATCTTAGGATGCGCTACTAGAGCGCTCTCTA
TTTCAGCCGTCCCCAACCTATGGCCGGAAACGTTTAAGACATCATCAACTCTACCAGTTATCCAGTAATATCC
ATCTTCATCTCTTCTGGCACCATCACCAGAAAAATACATGTTTTTAAAGGTGCTGAAATATGTTTGCTCAAAT
CTTTCATGATCTCCAAAAAGTGTCCTTGCTTGTCCGGGCCAAGAATCTGTTATTACTAAGTTACCTTCTGTCG
CGCCCTCTTGTGGATGACCTTCATTATCAACTAAAGCAGGCTGAACCCCGAAAAATGGCCGTGTCGCCGAACC
TGCCTTCAATTCAATAGCCCCAGGCAGCGGTGTGATCATGAACCCGCCTGTTTCGGTCTGCCACCAGGTGTCA
ACCACTGGGCATTTTTCCTTACCGATTTTCTTCCAATACCACTCCCAAGCTTCAGGATTAATTGGTTCGCCCA
CCGACCCTAAAATCCTTAAGCTAGAACGGTCCGTACCCTCAATGGCTTTATCGCCCTCCGCCATCAATGCTCT
GATCGCTGTCGGAGCGGTATACAAGATGTTCACTTGATGTTTGTCCACTACTTGACACATTCTAGCGGGAGTT
GGCCAGTTAGGAACACCCTCAAACATCAATGTAGTAGCACCACATGCGAGTGGTCCGTATAGTAAGTAAGAGT
GACCAGTTACCCAACCCACATCTGCTGTACACCAGTAAATGTCACCTGGGTGATAGTCAAATACATACTTAAA
TGTTGTAGCTGCGTACACCAAATAACCGCCGGTTGTATGAAGCACACCTTTTGGCTTGCCGGTGGAACCTGAA
GTGTACAGAATAAATAGAGGATCTTCAGCATTCATAGCTTCCGGTTGATGCTCTGGGGATGCTTTCTCAATCA
AATCTCTCCACCACAGATCCCTACCTTCTTGCCAATCTATGTCACTACCGGTTCTTTTCAAAACGATTACGTG
TTCAACAGAAGTTACATTTGGATTCTTTAACGCATCATCGACATTCTTTTTCAATGGGATAGACCTGCCTGCT
CTTACTCCTTCGTCTGCTGTAATAACTAGGCGACTACTACTATCTATGATCCTGCCAGCTACAGCCTCGGGTG
AAAAACCACCGAAAATCACGGAATGTACAGCTCCTATCCGAGCGCATGCTAGCATCGCTACGGCGGCCTCTGG
AACCATAGGCATATAAATAGCAACAACGTCACCCTTTTTAATACCTAAGTCTAGTAATGTGTTTGCGAACCTG
CACACATCTCTGTGTAGCTCTCTGTACGAGATGTGCTTGGATTGTGATGTATCATCTCCTTCCCATATTATGG
CGGTTCTATCTCCGTTCTCTTGTAAATGGCGGTCTAGGCAATTTGCAGCCAAATTCAATGTTCCATCTTCATA
CCATTTAATACTGACATTTCCAGGTGCAAATGAGGTGTTTTTAACCTTCTGATAGGGTGTGATCCAATCCAGA
ATCTTACCCTGCTCTCCCCAAAACGTGTCAGGATCATTGATAGATTGCTTATATTTTGTCTCATATTGTTCGG
GATTAATTAAGCACCGATCTGCTATATTAGCAGGAATTGCATGTTTGTGGGTCTGGCTCAT
Amino acid
Salmonella enterica acetyl CoA synthetase, feedback inhibition
resistant mutant
SEQ ID NO: 16
MSQTHKHAIPANIADRCLINPEQYETKYKQSINDPDTFWGEQGKILDWITPYQKVKNTSFAPGNVSIKWYEDG
TLNLAANCLDRHLQENGDRTAIIWEGDDTSQSKHISYRELHRDVCRFANTLLDLGIKKGDVVAIYMPMVPEAA
VAMLACARIGAVHSVIFGGFSPEAVAGRIIDSSSRLVITADEGVRAGRSIPLKKNVDDALKNPNVTSVEHVIV
LKRTGSDIDWQEGRDLWWRDLIEKASPEHQPEAMNAEDPLFILYTSGSTGKPKGVLHTTGGYLVYAATTFKYV
FDYHPGDIYWCTADVGWVTGHSYLLYGPLACGATTLMFEGVPNWPTPARMCQVVDKHQVNILYTAPTAIRALM
AEGDKAIEGTDRSSLRILGSVGEPINPEAWEWYWKKIGKEKCPVVDTWWQTETGGFMITPLPGAIELKAGSAT
RPFFGVQPALVDNEGHPQEGATEGNLVITDSWPGQARTLFGDHERFEQTYFSTFKNMYFSGDGARRDEDGYYW
ITGRVDDVLNVSGHRLGTAEIESALVAHPKIAEAAVVGIPHAIKGQAIYAYVTLNHGEEPSPELYAEVRNWVR
KEIGPLATPDVLHWTDSLPKTRSGKIMRRILRKIAAGDTSNLGDTSTLADPGVVEKPLEEKQAIAMPS
Native sequence DNA
Saccharomyces cerevisiae FDC1
SEQ ID NO: 17
ATGAGGAAGCTAAATCCAGCTTTAGAATTTAGAGACTTTATCCAGGTCTTAAAAGATGAAGATGACTTAATCG
AAATTACCGAAGAGATTGATCCAAATCTCGAAGTAGGTGCAATTATGAGGAAGGCCTATGAATCCCACTTACC
AGCCCCGTTATTTAAAAATCTCAAAGGTGCTTCGAAGGATCTTTTCAGCATTTTAGGTTGCCCAGCCGGTTTG
AGAAGTAAGGAGAAAGGAGATCATGGTAGAATTGCCCATCATCTGGGGCTCGACCCAAAAACAACTATCAAGG
AAATCATAGATTATTTGCTGGAGTGTAAGGAGAAGGAACCTCTCCCCCCAATCACTGTTCCTGTGTCATCTGC
ACCTTGTAAAACACATATACTTTCTGAAGAAAAAATACATCTACAAAGCCTGCCAACACCATATCTACATGTT
TCAGACGGTGGCAAGTACTTACAAACGTACGGAATGTGGATTCTTCAAACTCCAGATAAAAAATGGACTAATT
GGTCAATTGCTAGAGGTATGGTTGTAGATGACAAGCATATCACTGGTCTGGTAATTAAACCACAACATATTAG
ACAAATTGCTGACTCTTGGGCAGCAATTGGAAAAGCAAATGAAATTCCTTTCGCGTTATGTTTTGGCGTTCCC
CCAGCAGCTATTTTAGTTAGTTCCATGCCAATTCCTGAAGGTGTTTCTGAATCGGATTATGTTGGCGCAATCT
TGGGTGAGTCGGTTCCAGTAGTAAAATGTGAGACCAACGATTTAATGGTTCCTGCAACGAGTGAGATGGTATT
TGAGGGTACTTTGTCCTTAACAGATACACATCTGGAAGGCCCATTTGGTGAGATGCATGGATATGTTTTCAAA
AGCCAAGGTCATCCTTGTCCATTGTACACTGTCAAGGCTATGAGTTACAGAGACAATGCTATTCTACCTGTTT
CGAACCCCGGTCTTTGTACGGATGAGACACATACCTTGATTGGTTCACTAGTGGCTACTGAGGCCAAGGAGCT
GGCTATTGAATCTGGCTTGCCAATTCTGGATGCCTTTATGCCTTATGAGGCTCAGGCTCTTTGGCTTATCTTA
AAGGTGGATTTGAAAGGGCTGCAAGCATTGAAGACAACGCCTGAAGAATTTTGTAAGAAGGTAGGTGATATTT
ACTTTAGGACAAAAGTTGGTTTTATAGTCCATGAAATAATTTTGGTGGCAGATGATATCGACATATTTAACTT
CAAAGAAGTCATCTGGGCCTACGTTACAAGACATACACCTGTTGCAGATCAGATGGCTTTTGATGATGTCACT
TCTTTTCCTTTGGCTCCCTTTGTTTCGCAGTCATCCAGAAGTAAGACTATGAAAGGTGGAAAGTGCGTTACTA
ATTGCATATTTAGACAGCAATATGAGCGCAGTTTTGACTACATAACTTGTAATTTTGAAAAGGGATATCCAAA
AGGATTAGTTGACAAAGTAAATGAAAATTGGAAAAGGTACGGATATAAATAA
Native sequence amino acid
Saccharomyces cerevisiae FDC1
SEQ ID NO: 18
MRKLNPALEFRDFIQVLKDEDDLIEITEEIDPNLEVGAIMRKAYESHLPAPLFKNLKGASKDLFSILGCPAGL
RSKEKGDHGRIAHHLGLDPKTTIKEIIDYLLECKEKEPLPPITVPVSSAPCKTHILSEEKIHLQSLPTPYLHV
SDGGKYLQTYGMWILQTPDKKWTNWSIARGMVVDDKHITGLVIKPQHIRQIADSWAAIGKANEIPFALCFGVP
PAAILVSSMPIPEGVSESDYVGAILGESVPVVKCETNDLMVPATSEMVFEGTLSLTDTHLEGPFGEMHGYVFK
SQGHPCPLYTVKAMSYRDNAILPVSNPGLCTDETHTLIGSLVATEAKELAIESGLPILDAFMPYEAQALWLIL
KVDLKGLQALKTTPEEFCKKVGDIYFRTKVGFIVHEIILVADDIDIFNFKEVIWAYVTRHTPVADQMAFDDVT
SFPLAPFVSQSSRSKTMKGGKCVTNCIFRQQYERSFDYITCNFEKGYPKGLVDKVNENWKRYGYK
Native sequence DNA
Saccharomyces cerevisiae PAD1
SEQ ID NO: 19
ATGCTCCTATTTCCAAGAAGAACTAATATAGCCTTTTTCAAAACAACAGGCATTTTTGCTAATTTTCCTTTGC
TAGGTAGAACCATTACAACTTCACCATCTTTCCTTACACATAAACTGTCAAAGGAAGTAACCAGGGCATCAAC
TTCGCCTCCAAGACCAAAGAGAATTGTTGTCGCAATTACTGGTGCGACTGGTGTTGCACTGGGAATCAGACTT
CTACAAGTGCTAAAAGAGTTGAGCGTAGAAACCCATTTGGTGATTTCAAAATGGGGTGCAGCAACAATGAAAT
ATGAAACAGATTGGGAACCGCATGACGTGGCGGCCTTGGCAACCAAGACATACTCTGTTCGTGATGTTTCTGC
ATGCATTTCGTCCGGATCTTTCCAGCATGATGGTATGATTGTTGTGCCCTGTTCCATGAAATCACTAGCTGCT
ATTAGAATCGGTTTTACAGAGGATTTAATTACAAGAGCTGCCGATGTTTCGATTAAAGAGAATCGTAAGTTAC
TACTGGTTACTCGGGAAACCCCTTTATCTTCCATCCATCTTGAAAACATGTTGTCTTTATGCAGGGCAGGTGT
TATAATTTTTCCTCCGGTACCTGCGTTTTATACAAGACCCAAGAGCCTTCATGACCTATTAGAACAAAGTGTT
GGCAGGATCCTAGACTGCTTTGGCATCCACGCTGACACTTTTCCTCGTTGGGAAGGAATAAAAAGCAAGTAA
Amino acid
Saccharomyces cerevisiae PAD1
SEQ ID NO: 20
MLLFPRRTNIAFFKTTGIFANFPLLGRTITTSPSFLTHKLSKEVTRASTSPPRPKRIVVAITGATGVALGIRL
LQVLKELSVETHLVISKWGAATMKYETDWEPHDVAALATKTYSVRDVSACISSGSFQHDGMIVVPCSMKSLAA
IRIGFTEDLITRAADVSIKENRKLLLVTRETPLSSIHLENMLSLCRAGVIIFPPVPAFYTRPKSLHDLLEQSV
GRILDCFG1HADTFPRWEGIKSK
Native sequence DNA
Saccharomyces cerevisiae ARO10
SEQ ID NO: 21
ATGGCACCTGTTACAATTGAAAAGTTCGTAAATCAAGAAGAACGACACCTTGTTTCCAACCGATCAGCAACAA
TTCCGTTTGGTGAATACATATTTAAAAGATTGTTGTCCATCGATACGAAATCAGTTTTCGGTGTTCCTGGTGA
CTTCAACTTATCTCTATTAGAATATCTCTATTCACCTAGTGTTGAATCAGCTGGCCTAAGATGGGTCGGCACG
TGTAATGAACTGAACGCCGCTTATGCGGCCGACGGATATTCCCGTTACTCTAATAAGATTGGCTGTTTAATAA
CCACGTATGGCGTTGGTGAATTAAGCGCCTTGAACGGTATAGCCGGTTCGTTCGCTGAAAATGTCAAAGTTTT
GCACATTGTTGGTGTGGCCAAGTCCATAGATTCGCGTTCAAGTAACTTTAGTGATCGGAACCTACATCATTTG
GTCCCACAGCTACATGATTCAAATTTTAAAGGGCCAAATCATAAAGTATATCATGATATGGTAAAAGATAGAG
TCGCTTGCTCGGTAGCCTACTTGGAGGATATTGAAACTGCATGTGACCAAGTCGATAATGTTATCCGCGATAT
TTACAAGTATTCTAAACCTGGTTATATTTTTGTTCCTGCAGATTTTGCGGATATGTCTGTTACATGTGATAAT
TTGGTTAATGTTCCACGTATATCTCAACAAGATTGTATAGTATACCCTTCTGAAAACCAATTGTCTGACATAA
TCAACAAGATTACTAGTTGGATATATTCCAGTAAAACACCTGCGATCCTTGGAGACGTACTGACTGATAGGTA
TGGTGTGAGTAACTTTTTGAACAAGCTTATCTGCAAAACTGGGATTTGGAATTTTTCCACTGTTATGGGAAAA
TCTGTAATTGATGAGTCAAACCCAACTTATATGGGTCAATATAATGGTAAAGAAGGTTTAAAACAAGTCTATG
AACATTTTGAACTGTGCGACTTGGTCTTGCATTTTGGAGTCGACATCAATGAAATTAATAATGGGCATTATAC
TTTTACTTATAAACCAAATGCTAAAATCATTCAATTTCATCCGAATTATATTCGCCTTGTGGACACTAGGCAG
GGCAATGAGCAAATGTTCAAAGGAATCAATTTTGCCCCTATTTTAAAAGAACTATACAAGCGCATTGACGTTT
CTAAACTTTCTTTGCAATATGATTCAAATGTAACTCAATATACGAACGAAACAATGCGGTTAGAAGATCCTAC
CAATGGACAATCAAGCATTATTACACAAGTTCACTTACAAAAGACGATGCCTAAATTTTTGAACCCTGGTGAT
GTTGTCGTTTGTGAAACAGGCTCTTTTCAATTCTCTGTTCGTGATTTCGCGTTTCCTTCGCAATTAAAATATA
TATCGCAAGGATTTTTCCTTTCCATTGGCATGGCCCTTCCTGCCGCCCTAGGTGTTGGAATTGCCATGCAAGA
CCACTCAAACGCTCACATCAATGGTGGCAACGTAAAAGAGGACTATAAGCCAAGATTAATTTTGTTTGAAGGT
GACGGTGCAGCACAGATGACAATCCAAGAACTGAGCACCATTCTGAAGTGCAATATTCCACTAGAAGTTATCA
TTTGGAACAATAACGGCTACACTATTGAAAGAGCCATCATGGGCCCTACCAGGTCGTATAACGACGTTATGTC
TTGGAAATGGACCAAACTATTTGAAGCATTCGGAGACTTCGACGGAAAGTATACTAATAGCACTCTCATTCAA
TGTCCCTCTAAATTAGCACTGAAATTGGAGGAGCTTAAGAATTCAAACAAAAGAAGCGGGATAGAACTTTTAG
AAGTCAAATTAGGCGAATTGGATTTCCCCGAACAGCTAAAGTGCATGGTTGAAGCAGCGGCACTTAAAAGAAA
TAAAAAATAG
Native sequence DNA
Saccharomyces cerevisiae PDC5
SEQ ID NO: 22
ATGTCTGAAATAACCTTAGGTAAATATTTATTTGAAAGATTGAGCCAAGTCAACTGTAACACCGTCTTCGGTT
TGCCAGGTGACTTTAACTTGTCTCTTTTGGATAAGCTTTATGAAGTCAAAGGTATGAGATGGGCTGGTAACGC
TAACGAATTGAACGCTGCCTATGCTGCTGATGGTTACGCTCGTATCAAGGGTATGTCCTGTATTATTACCACC
TTCGGTGTTGGTGAATTGTCTGCTTTGAATGGTATTGCCGGTTCTTACGCTGAACATGTCGGTGTTTTGCACG
TTGTTGGTGTTCCATCCATCTCTTCTCAAGCTAAGCAATTGTTGTTGCATCATACCTTGGGTAACGGTGACTT
CACTGTTTTCCACAGAATGTCTGCCAACATTTCTGAAACCACTGCCATGATCACTGATATTGCTAACGCTCCA
GCTGAAATTGACAGATGTATCAGAACCACCTACACTACCCAAAGACCAGTCTACTTGGGTTTGCCAGCTAACT
TGGTTGACTTGAACGTCCCAGCCAAGTTATTGGAAACTCCAATTGACTTGTCTTTGAAGCCAAACGACGCTGA
AGCTGAAGCTGAAGTTGTTAGAACTGTTGTTGAATTGATCAAGGATGCTAAGAACCCAGTTATCTTGGCTGAT
GCTTGTGCTTCTAGACATGATGTCAAGGCTGAAACTAAGAAGTTGATGGACTTGACTCAATTCCCAGTTTACG
TCACCCCAATGGGTAAGGGTGCTATTGACGAACAACACCCAAGATACGGTGGTGTTTACGTTGGTACCTTGTC
TAGACCAGAAGTTAAGAAGGCTGTAGAATCTGCTGATTTGATATTGTCTATCGGTGCTTTGTTGTCTGATTTC
AATACCGGTTCTTTCTCTTACTCCTACAAGACCAAAAATATCGTTGAATTCCACTCTGACCACATCAAGATCA
GAAACGCCACCTTCCCAGGTGTTCAAATGAAATTTGCCTTGCAAAAATTGTTGGATGCTATTCCAGAAGTCGT
CAAGGACTACAAACCTGTTGCTGTCCCAGCTAGAGTTCCAATTACCAAGTCTACTCCAGCTAACACTCCAATG
AAGCAAGAATGGATGTGGAACCATTTGGGTAACTTCTTGAGAGAAGGTGATATTGTTATTGCTGAAACCGGTA
CTTCCGCCTTCGGTATTAACCAAACTACTTTCCCAACAGATGTATACGCTATCGTCCAAGTCTTGTGGGGTTC
CATTGGTTTCACAGTCGGCGCTCTATTGGGTGCTACTATGGCCGCTGAAGAACTTGATCCAAAGAAGAGAGTT
ATTTTATTCATTGGTGACGGTTCTCTACAATTGACTGTTCAAGAAATCTCTACCATGATTAGATGGGGTTTGA
AGCCATACATTTTTGTCTTGAATAACAACGGTTACACCATTGAAAAATTGATTCACGGTCCTCATGCCGAATA
TAATGAAATTCAAGGTTGGGACCACTTGGCCTTATTGCCAACTTTTGGTGCTAGAAACTACGAAACCCACAGA
GTTGCTACCACTGGTGAATGGGAAAAGTTGACTCAAGACAAGGACTTCCAAGACAACTCTAAGATTAGAATGA
TTGAAGTTATGTTGCCAGTCTTTGATGCTCCACAAAACTTGGTTAAACAAGCTCAATTGACTGCCGCTACTAA
CGCTAAACAATAA

As is evident from the foregoing description, certain aspects of the present disclosure are not limited by the particular details of the examples provided herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

1. A microorganism of the genus Saccharomyces, comprising a disrupted gene encoding an enzyme involved in the degradation of phenylpyruvate to phenylacetaldehyde.

2. The microorganism of claim 1, wherein the disrupted gene encoding an enzyme involved in the degradation of phenylpyruvate to phenylacetaldehyde is selected from the group consisting of ARO10, PDCS, and combinations thereof.

3. The microorganism of claim 1, where the gene encoding an enzyme involved in the degradation of phenylpyruvate to phenylacetaldehyde is disrupted by partial or total deletion.

4. The microorganism of claim 1, further comprising a recombinant gene encoding a 4-coumaric acid:Coenzyme A ligase from Arabidopsis thaliana.

5. The microorganism of claim 4, wherein the recombinant gene encoding a 4-coumaric acid:Coenzyme A ligase from Arabidopsis thaliana is At4CL1.

6. The microorganism of claim 4, wherein the recombinant gene encoding a 4-coumaric acid:Coenzyme A ligase has at least 90%, 95%, or 99% sequence identity to any one of SEQ. ID. NOs: 1 and 12.

7. The microorganism of claim 4, wherein the recombinant gene encoding a 4-coumaric acid:Coenzyme A ligase has at least 98% or 99% sequence identity to any one of SEQ. ID. NOs: 1 and 12.

8. The microorganism of claim 4, wherein the recombinant gene encoding a 4-coumaric acid:Coenzyme A ligase has a sequence according to any one of SEQ. ID. NOs: 1 and 12.

9. The microorganism of claim 1, further comprising a recombinant gene encoding a Vitis vinifera stilbene synthase.

10. The microorganism of claim 9, where the Vitis vinifera stilbene synthase gene has at least 90%, 95%, or 99% sequence identity to any one of SEQ. ID. NOs.: 3, 4, 5, 6, 7, and 8.

11. The microorganism of claim 9, wherein the Vitis vinifera stilbene synthase gene has at least 98%, or 99% sequence identity to any one of SEQ. ID. NOs.: 3, 4, 5, 6, 7, and 8.

12. The microorganism of claim 9, wherein the Vitis vinifera stilbene synthase gene has a nucleotide sequence selected from the group consisting of SEQ. ID. NOs.: 3, 4, 5, 6, 7, and 8.

13. The microorganism of claim 1, further comprising a recombinant gene encoding a feedback inhibition-resistant mutant of an acetyl-CoA carboxylase.

14. The microorganism of claim 13, wherein the gene encoding a feedback inhibition-resistant mutant of an acetyl-CoA carboxylase has at least 90%, 95%, or 99% sequence identity to SEQ. ID. NO: 10.

15. The microorganism of claim 13, wherein the gene encoding a feedback inhibition-resistant mutant of an acetyl-CoA carboxylase has at least 98%, or 99% sequence identity to SEQ. ID. NO: 10.

16. The microorganism of claim 13, wherein the gene encoding a feedback inhibition-resistant mutant of an acetyl-CoA carboxylase comprises a nucleotide sequence according to SEQ ID NO: 10.

17. The microorganism of claim 13, wherein the feedback inhibition-resistant mutant of an acetyl-CoA carboxylase comprises an amino acid sequence according to SEQ ID NO:

11.

18. The microorganism of claim 1, wherein the microorganism is Saccharomyces cerevisiae.

19. A method of producing resveratrol using a recombinant Saccharomyces cell, the method comprising:
(i) cultivating a recombinant Saccharomyces cell in a medium;
(ii) adding 4-coumaric acid to the medium to initiate the bioconversion of 4-coumaric acid to resveratrol; and
(iii) extracting resveratrol from at least one of the recombinant cell and medium, wherein the recombinant Saccharomyces cell has been transformed to disrupt a gene encoding an enzyme involved in the degradation of phenylpyruvate to phenylacetaldehyde.

20. The method of claim 19, wherein the disrupted gene encoding an enzyme involved in the degradation of phenylpyruvate to phenylacetaldehyde is selected from the group consisting of ARO10, PDCS, and combinations thereof.

21. The method of claim 19, wherein the Saccharomyces cell has been further transformed with a nucleic acid construct encoding a 4-coumaric acid:Coenzyme A ligase from Arabidopsis thaliana.

22. The method of claim 21, wherein the 4-coumaric acid:Coenzyme A ligase from Arabidopsis thaliana comprises an amino acid sequence according to SEQ ID NO: 2.

23. The method of claim 19, wherein the Saccharomyces cell has been further transformed with a nucleic acid construct encoding a stilbene synthase from Vitis vinifera.

24. The method of claim 23, wherein the stilbene synthase from Vitis vinifera comprises an amino sequence according to SEQ ID NO: 9.

25. The method of claim 19, wherein the Saccharomyces cell has been further transformed with a nucleic acid construct encoding a feedback inhibition-resistant mutant of an acetyl-CoA carboxylase.

26. The method of claim 25, wherein the feedback inhibition-resistant mutant of an acetyl-CoA carboxylase comprises an amino acid sequence according to SEQ ID NO: 11.

27. The method of claim 19, wherein the recombinant Saccharomyces cell is Saccharomyces cerevisiae.


SEQUENCE LISTING (22 sequences)

SEQ ID NO: 1 - DNA, 1686 bp, Artificial Sequence
  Synthetic
atggcgccacaagaacaagcagtttctcaggtgatggagaaacagagcaacaacaacaacagtgacgtcattttccgatcaaagttaccggatatttacatcccgaaccacctatctctccacgactacatcttccaaaacatctccgaattcgccactaagccttgcctaatcaacggaccaaccggccacgtgtacacttactccgacgtccacgtcatctcccgccaaatcgccgccaattttcacaaactcggcgttaaccaaaacgacgtcgtcatgctcctcctcccaaactgtcccgaattcgtcctctctttcctcgccgcctccttccgcggcgcaaccgccaccgccgcaaaccctttcttcactccggcggagatagctaaacaagccaaagcctccaacaccaaactcataatcaccgaagctcgttacgtcgacaaaatcaaaccacttcaaaacgacgacggagtagtcatcgtctgcatcgacgacaacgaatccgtgccaatccctgaaggctgcctccgcttcaccgagttgactcagtcgacaaccgaggcatcagaagtcatcgactcggtggagatttcaccggacgacgtggtggcactaccttactcctctggcacgacgggattaccaaaaggagtgatgctgactcacaagggactagtcacgagcgttgctcagcaagtcgacggcgagaacccgaatctttatttccacagcgatgacgtcatactctgtgttttgcccatgtttcatatctacgctttgaactcgatcatgttgtgtggtcttagagttggtgcggcgattctgataatgccgaagtttgagatcaatctgctatgggagctgatccagaggtgtaaagtgacggtggctccgatggttccgccgattgtgttggccattgcgaagtcttcggaaacggagaagtatgatttgagctcgataagagtggtgaaatctggtgctgctcctcttggtaaagaacttgaagatgccgttaatgccaagtttcctaatgccaaactcggtcagggatacggaatgacggaagcaggtccagtgctagcaatgtcgttaggttttgcaaaggaaccttttccggttaagtcaggagcttgtggtactgttgtaagaaatgctgagatgaaaatagttgatccagacaccggagattctctttcgaggaatcaacccggtgagatttgtattcgtggtcaccagatcatgaaaggttacctcaacaatccggcagctacagcagaaaccattgataaagacggttggcttcatactggagatattggattgatcgatgacgatgacgagcttttcatcgttgatcgattgaaagaacttatcaagtataaaggttttcaggtagctccggctgagctagaggctttgctcatcggtcatcctgacattactgatgttgctgttgtcgcaatgaaagaagaagcagctggtgaagttcctgttgcatttgtggtgaaatcgaaggattcggagttatcagaagatgatgtgaagcaattcgtgtcgaaacaggttgtgttttacaagagaatcaacaaagtgttcttcactgaatccattcctaaagctccatcagggaagatattgaggaaagatctgagggcaaaactagcaaatggattgtga

SEQ ID NO: 2 - protein, 561 aa, Artificial sequence
  Synthetic
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLWELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 3 - DNA, 1179 bp, Artificial sequence
  Synthetic
atggcttctgttgaggaatttaggaatgctcaacgtgccaagggacccgccactattctggctataggtactgccaccccagatcattgcgtatatcaatcggattacgctgactactacttcaaggttaccaaaagtgagcacatgacagccttgaagaagaagtttaaccgtatatgcgataagtcaatgatcaagaaaagatacattcacttgacagaagaaatgttagaggaacatccaaatataggcgcttacatggctccatcgttaaacatccgtcaggaaatcattacagctgaagtacccaaattaggtaaagaggctgcattgaaagccctaaaagaatggggccaacctaaatccaaaattactcatttggtattctgtaccacaagcggcgttgaaatgcctggagctgactataaacttgccaacctactgggcttggaaccttccgtccgtagggtaatgctttaccaccaaggttgttatgctggtgggacagtcttgaggacggctaaggacttagccgaaaataatgctggggcacgggttctagttgtatgttcggaaattacggttgtaacttttcgtggtccatcagaagatgcattagattcgttggtcggtcaggcattatttggcgatggctccgcagcagtcatcgtcggttcggatccagatattagtatagagcgccccttgttccaactcgtatccgcagctcaaacatttattccaaactccgcgggtgcgattgccgggaacttacgggaagtgggtttaacctttcacctctggccaaatgttcctacccttatttccgaaaacgttgagaaatgcctaacacaagctttcgatcctctaggaatctcggattggaatagcttgttctggattgcccatccaggtggtcctgccattcttgatgcggttgaggctaaattgaacctagacaagaagaagttggaagccacaagacatgtactgtcagaatatggaaatatgagttctgcctgtgtcttattcatactcgacgaaatgagaaagaagtccttaaagggcgaaagagctactaccggcgaaggactagattggggagttttgtttggtttcggtcctggattgacaattgaaacagttgttttgcatagtattcccatggttaccaattaa

SEQ ID NO: 4 - DNA, 1179 bp, Artificial sequence
  Synthetic
atggctagcgtggaggaatttaggaatgcacagagagcgaaagggcctgctaccattttagcaatcggtactgcgactccagatcattgtgtataccaaagtgattatgcagactattatttcaaggtcaccaagtctgaacacatgaccgcattaaagaagaagtttaatagaatatgcgataagagcatgatcaagaaacgttatattcacttgacggaagaaatgttggaagaacatcctaatataggtgcttacatggcaccctctttgaatatcagacaggaaataattacggcagaagttcccaaattgggaaaagaggctgccttgaaggctttaaaagaatggggtcagcccaaatctaaaattacccacttagtattttgtacgacatcaggcgtcgaaatgccaggtgcggattacaaattagccaatttgttaggtttggaaccgtcagttagacgtgttatgttgtaccatcaaggatgctatgccggtgggacggttctgagaacagcgaaagatctagctgagaataacgcaggcgcaagagtattggtagtctgttccgaaataactgttgtcactttcagaggcccaagtgaggacgcgttggactcattagttggtcaggcactgtttggcgatggttctgccgctgtaattgtcggtagcgaccctgatataagtattgaaagacccctgttccaattggtttcagcagcacaaacttttattcctaatagtgctggtgctatcgctggtaatttaagagaagttggcttaacatttcatttgtggcctaatgttccaaccctgataagcgaaaacgtagagaaatgtcttacccaagcgttcgacccattaggaattagtgattggaactctcttttctggatcgcacacccaggaggcccagctatattagacgcagttgaagctaagttaaatttagataagaagaaattggaggcaacaagacatgtgttatccgagtacggaaatatgtcatcagcatgtgtgttgtttatattggacgagatgagaaagaagagtcttaagggagagagagctaccacaggagagggattggattggggtgtcttatttggttttggtccaggtctaacaattgaaacagtagtgttacactctattccaatggtcacaaattaa

SEQ ID NO: 5 - DNA, 1179 bp, Artificial sequence
  Synthetic
atggcatccgtggaagaatttagaaacgcacagagggcaaaaggtccagcaaccatactagctatcggcacagctacccctgatcattgcgtctatcagtcggactacgctgattattattttaaggttaccaaatcagaacacatgaccgcattgaagaagaagtttaacagaatatgtgacaaatcaatgattaagaagcgctatattcatctaactgaggagatgctggaggaacatccaaatattggtgcgtacatggcaccatccctaaacattcgccaagagattattacggctgaagttcccaagttaggcaaggaagcagctctgaaggcattaaaggagtggggccagcctaagagcaaaatcactcatcttgtattttgtacgacctctggtgtggaaatgcctggagctgactataaattagcgaacttgttgggcctagagccaagtgttagaagggtgatgctgtatcatcagggttgttatgcaggtggtactgtcttgaggacagccaaggatctggctgaaaataatgctggcgccagagtactcgtagtatgcagtgagatcaccgtcgtcacatttaggggaccatctgaagatgctttggattctctcgttggccaggctttattcggcgatggttccgctgctgtgatagtcggctcggatcctgacatatccatcgaacgccccttgtttcaattagttagcgcagcgcagacctttatacctaactcggccggggcaatagcaggtaatttgcgtgaagtcggattgacttttcatttgtggcctaacgtccccacgttgatttcagaaaatgtcgaaaagtgtttaacgcaagcattcgatcctctaggtatatctgattggaatagcctcttctggattgcacatcctggcgggcctgctattctggacgcggtcgaggctaagttaaatttggataagaagaagctggaagccaccagacatgtcctgtctgagtacgggaatatgtcaagtgcatgtgtgctctttatactggacgagatgaggaagaaatcgttaaagggtgagagagctactacgggtgaaggattagattggggcgtattattcggcttcggtccggggctcactatcgaaacagtagtcctgcatagtatccccatggtcaccaattga

SEQ ID NO: 6 - DNA, 1179 bp, Artificial sequence
  Synthetic
atggcctcagtagaagagtttcgtaatgctcaaagagccaagggcccagctacaattttagctataggcaccgctacgccagatcattgtgtttaccaatccgattacgcagattactatttcaaggtcacaaagagcgaacacatgactgccttaaagaagaaatttaaccgtatctgtgacaaatctatgatcaagaagcgttacatacatttgactgaagagatgttagaggagcaccctaacattggtgcctacatggcaccgtcgttaaatatccgtcaagaaattattacagctgaggtcccaaagttaggtaaggaagctgctcttaaagccttgaaggaatggggtcaacctaagagtaaaattacacatttggtcttttgtaccacttccggcgttgaaatgcctggcgccgattacaagttagctaacctattaggtctggaaccaagcgttcgtcgcgtaatgttataccatcagggatgttatgcaggtggtactgtattaaggaccgcaaaagacttggcagaaaataacgcgggcgccagagtattggtcgtgtgtagcgaaattacggttgtaacattcaggggtccatcagaggacgcactggacagtctcgtagggcaagcactatttggtgatggaagcgctgcggtcattgttggtagcgacccagacatatcaattgaaagacctcttttccaacttgtctctgctgcccaaacttttattccgaatagcgccggggctatcgcgggtaatcttagagaagtgggactgacgtttcatttatggccaaatgtgcccacacttataagcgaaaatgtcgaaaaatgtcttacgcaggcattcgatcctcttggtatatcggattggaactctctcttttggatcgcccatccaggtggtcctgcaattctggatgctgtagaagcaaaactaaacctggacaagaagaaactggaagctacaagacatgtcttgtcggaatacgggaacatgagttcggcatgtgtactttttattttagatgagatgcgtaaaaagtctctgaaaggtgagcgtgcaacaaccggtgaaggtttggactggggtgtcttgttcggattcggtcccggcttaaccatcgaaactgtagttctacattctattccaatggttactaattaa

SEQ ID NO: 7 - DNA, 1179 bp, Artificial sequence
  Synthetic
atggcttcagtcgaggagtttagaaatgctcagagggccaagggtcctgccacaatattagctataggtactgccaccccagatcactgtgtctatcaaagtgactatgctgactattattttaaagtcacaaaaagtgagcacatgactgcattgaaaaagaaattcaataggatatgtgataaatcaatgatcaaaaagagatacattcatctaactgaggaaatgttagaagagcatccaaatattggtgcatatatggctccatccttaaatatcagacaggaaataataaccgctgaggtgcctaaactgggtaaagaagctgcattaaaagcattaaaagaatggggtcagcctaaatcaaagattacgcatctagtattttgcacaacgtctggtgtcgaaatgcctggagccgattacaaactagcaaatttactaggtcttgaaccttctgtccgtcgagtaatgttataccaccaaggttgctacgcaggcggaaccgttctaaggactgccaaggacttggcagaaaataacgctggtgcaagggttttagtggtttgttctgaaatcactgtagtcacatttaggggtccctctgaagatgcattagactctttagttgggcaagcactgttcggggatgggtctgcggccgttatagtaggttcagatcctgacatttctatcgaaaggcctctgtttcaactggtatctgctgcccaaacttttattcctaacagcgctggtgcaatcgccgggaacctccgagaagtaggtcttacatttcatctatggcctaatgtccctactttgatttccgagaatgtagagaaatgcctgactcaggcctttgatcctttgggcatatctgattggaactcactattttggattgcacaccccggaggtcccgcaattttggatgccgtggaggctaaattaaatttagataagaagaaactcgaagcaactagacatgtattatcagagtacggcaatatgtctagtgcttgtgttttatttattttagacgaaatgcgtaaaaagtctttaaagggagagagggctactacaggagaaggattagattggggtgttttgtttggtttcggacccggtttaacgatcgaaacagttgttctgcatagtatccctatggtgaccaattga

SEQ ID NO: 8 - DNA, 1179 bp, Artificial sequence
  Synthetic
atggcatcggtagaagagttcagaaatgcacagagggctaaaggccctgccacaatcctagcaattggtactgcaactcccgatcattgcgtttatcaaagtgattatgccgactattattttaaagttacgaaatcagaacacatgactgctcttaaaaagaaattcaacagaatatgtgacaagagtatgattaaaaagagatacattcacttgacagaagagatgttggaggagcatcctaatatcggcgcttacatggcaccttcattgaacattcgtcaagaaataattactgccgaggttcctaaactcggcaaagaagcagcacttaaggcacttaaggaatggggtcagccaaagtcaaagatcacacatttggtcttttgtacaacctctggagttgagatgccaggcgctgattataaattggctaatcttttaggattagagccaagtgttaggcgggtgatgctatatcaccaaggttgttatgcaggtggtactgttttgaggacagccaaggatctggccgaaaataatgctggggccagagtcctggttgtttgctccgagataactgttgttacatttcgcgggccttcagaagatgcactggattctcttgtgggacaggcgctgtttggtgatgggtccgctgccgtgatcgtaggctctgatccagatatatcaattgagaggcctttatttcagttggtgtctgccgctcagacattcatccctaattccgcgggagcgatagctggtaatctaagagaggttggcttgacatttcacttatggcctaatgtgccaacattgatctctgagaacgtcgaaaagtgcctaacccaagcatttgacccattaggaattagcgactggaatagtttattttggatagcacaccctggaggtccggctatattggatgctgtggaagcaaagctaaatctggataagaagaagctagaagcaacaagacacgtactatctgaatacggaaatatgagcagtgcttgtgttctatttattcttgatgagatgcgtaaaaagagtttaaatggagaaagagccaccacaggtgaagggctagactggggcgttttatttggcttcggtccaggtctgacaatcgaaacggtcgtcttacactcaattccaatggttacaaattga

SEQ ID NO: 9 - protein, 392 aa, Artificial sequence
  Synthetic
MASVEEFRNAQRAKGPATILAIGTATPDHCVYQSDYADYYFKVTKSEHMTALKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLYHQGCYAGGTVLRTAKDLAENNAGARVLVVCSEITVVTFRGPSEDALDSLVGQALFGDGSAAVIVGSDPDISIERPLFQLVSAAQTFIPNSAGAIAGNLREVGLTFHLWPNVPTLISENVEKCLTQAFDPLGISDWNSLFWIAHPGGPAILDAVEAKLNLDKKKLEATRHVLSEYGNMSSACVLFILDEMRKKSLKGERATTGEGLDWGVLFGFGPGLTIETVVLHSIPMVTN

SEQ ID NO: 10 - DNA, 6702 bp, Artificial sequence
  Synthetic
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactagctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgttgctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 11 - protein, 2233 aa, Artificial sequence
  Synthetic
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLADGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVAVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 12 - DNA, 1671 bp, Artificial sequence
  Synthetic
atgactacgcaggatgttattgtcaatgatcaaaatgaccaaaagcaatgttcgaatgatgttatctttcgtagtagactccctgatatatacatacctaaccatctaccattgcatgattacatatttgaaaatatatcggaatttgctgctaagccatgcctaatcaatggtccaacaggtgaagtgtatacctatgctgatgttcatgttacttccaggaagctcgctgctggtttgcacaacttgggcgttaaacagcatgacgtcgttatgatattgctgccaaatagcccagaagtggtacttactttcttggccgcctcgtttattggcgccattacgacatccgcaaatcccttcttcacgcccgctgaaatttctaaacaagctaaagcatctgctgctaaattaatcgtcacacaaagtagatatgttgataagattaagaacttacaaaacgatggggtcttaattgtcacaaccgattctgatgctatccctgaaaattgtctgagattctctgagttaactcaatccgaagagcctagagtagacagtatacctgagaagatctctccagaagatgtggtggctttgccattttcctcaggtactaccggtctgccaaagggtgtgatgttgactcacaagggtttggtgacgtcagtagctcagcaagtagatggggagaaccctaatctgtatttcaatagagatgacgtcattttgtgcgtattacctatgttccatatttatgcattaaactcgattatgctatgctctctgcgagttggagcaactatattaatcatgccaaagtttgagataactctcttgttagaacaaattcagaggtgcaaggtcactgttgctatggtagtaccaccaatagtcctggcaatcgcaaagagtcctgaaaccgagaagtatgatttaagtagtgtgcggatggttaaatcaggcgctgcccctctaggtaaagaattagaagatgccatttccgctaaatttccgaatgcaaaattaggccaaggatatggcatgacggaagctggtccagttctagcaatgtctttggggtttgctaaagagccttttcccgtaaagagcggtgcctgtggcactgttgtgcgtaatgctgagatgaaaatactggatccagacacgggcgattcactaccacgcaataaaccaggcgagatatgtataaggggaaaccagattatgaaggggtatttgaacgatcccctggccaccgcctcaactatcgataaggacggatggttacacactggtgacgttgggtttattgacgatgatgatgaattattcatcgttgacagattaaaggaattgatcaaatacaaaggttttcaagtagctccagcagaactcgaaagccttttgattggacatccagagataaatgacgtcgcagtggtcgctatgaaagaagaggatgctggtgaagttcccgttgcatttgtagttagatcgaaggattccaacattagcgaggacgaaattaaacaatttgtaagcaaacaggttgtcttttataaaagaatcaataaagttttcttcactgactcaattccaaaggccccttctggtaaaatcctgcgtaaggacttgagggcacgattggctaatggcctcatgaattga

SEQ ID NO: 13 - protein, 556 aa, Artificial sequence
  Synthetic
MTTQDVIVNDQNDQKQCSNDVIFRSRLPDIYIPNHLPLHDYIFENISEFAAKPCLINGPTGEVYTYADVHVTSRKLAAGLHNLGVKQHDVVMILLPNSPEVVLTFLAASFIGAITTSANPFFTPAEISKQAKASAAKLIVTQSRYVDKIKNLQNDGVLIVTTDSDAIPENCLRFSELTQSEEPRVDSIPEKISPEDVVALPFSSGTTGLPKGVMLTHKGLVTSVAQQVDGENPNLYFNRDDVILCVLPMFHIYALNSIMLCSLRVGATILIMPKFEITLLLEQIQRCKVTVAMVVPPIVLAIAKSPETEKYDLSSVRMVKSGAAPLGKELEDAISAKFPNAKLGQGYGMTEAGPVLAMSLGFAKEPFPVKSGACGTVVRNAEMKILDPDTGDSLPRNKPGEICIRGNQIMKGYLNDPLATASTIDKDGWLHTGDVGFIDDDDELFIVDRLKELIKYKGFQVAPAELESLLIGHPEINDVAVVAMKEEDAGEVPVAFVVRSKDSNISEDEIKQFVSKQVVFYKRINKVFFTDSIPKAPSGKILRKDLRARLANGLMN

SEQ ID NO: 14 - DNA, 7732 bp, Artificial sequence
  Synthetic
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtttaaaaaaaatggcttctgttgaggaatttaggaatgctcaacgtgccaagggacccgccactattctggctataggtactgccaccccagatcattgcgtatatcaatcggattacgctgactactacttcaaggttaccaaaagtgagcacatgacagccttgaagaagaagtttaaccgtatatgcgataagtcaatgatcaagaaaagatacattcacttgacagaagaaatgttagaggaacatccaaatataggcgcttacatggctccatcgttaaacatccgtcaggaaatcattacagctgaagtacccaaattaggtaaagaggctgcattgaaagccctaaaagaatggggccaacctaaatccaaaattactcatttggtattctgtaccacaagcggcgttgaaatgcctggagctgactataaacttgccaacctactgggcttggaaccttccgtccgtagggtaatgctttaccaccaaggttgttatgctggtgggacagtcttgaggacggctaaggacttagccgaaaataatgctggggcacgggttctagttgtatgttcggaaattacggttgtaacttttcgtggtccatcagaagatgcattagattcgttggtcggtcaggcattatttggcgatggctccgcagcagtcatcgtcggttcggatccagatattagtatagagcgccccttgttccaactcgtatccgcagctcaaacatttattccaaactccgcgggtgcgattgccgggaacttacgggaagtgggtttaacctttcacctctggccaaatgttcctacccttatttccgaaaacgttgagaaatgcctaacacaagctttcgatcctctaggaatctcggattggaatagcttgttctggattgcccatccaggtggtcctgccattcttgatgcggttgaggctaaattgaacctagacaagaagaagttggaagccacaagacatgtactgtcagaatatggaaatatgagttctgcctgtgtcttattcatactcgacgaaatgagaaagaagtccttaaagggcgaaagagctactaccggcgaaggactagattggggagttttgtttggtttcggtcctggattgacaattgaaacagttgttttgcatagtattcccatggttaccaattaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaaggtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 15 - DNA, 1959 bp, Artificial sequence
  Synthetic
ctatgatggcattgcaatggcttgcttctcttcgagcggtttttccacaacgccaggatctgcgagagtggacgtgtcacctaaattacttgtatctccggcggcgatttttcgcaggattctcctcataattttcccactccttgtttttggtagagagtctgtccaatgtaaaacatctggagtagccaaaggcccaatctcttttcgaacccagttccttacctccgcatacaattctggagaaggctcttcaccatgattgagtgtcacataagcatatatagcttgccctttgatagcatgagggatgcccacaacagccgcttcagctatcttaggatgcgctactagagcgctctctatttcagccgtccccaacctatggccggaaacgtttaagacatcatcaactctaccagttatccagtaatatccatcttcatctcttctggcaccatcaccagaaaaatacatgtttttaaaggtgctgaaatatgtttgctcaaatctttcatgatctccaaaaagtgtccttgcttgtccgggccaagaatctgttattactaagttaccttctgtcgcgccctcttgtggatgaccttcattatcaactaaagcaggctgaaccccgaaaaatggccgtgtcgccgaacctgccttcaattcaatagccccaggcagcggtgtgatcatgaacccgcctgtttcggtctgccaccaggtgtcaaccactgggcatttttccttaccgattttcttccaataccactcccaagcttcaggattaattggttcgcccaccgaccctaaaatccttaagctagaacggtccgtaccctcaatggctttatcgccctccgccatcaatgctctgatcgctgtcggagcggtatacaagatgttcacttgatgtttgtccactacttgacacattctagcgggagttggccagttaggaacaccctcaaacatcaatgtagtagcaccacatgcgagtggtccgtatagtaagtaagagtgaccagttacccaacccacatctgctgtacaccagtaaatgtcacctgggtgatagtcaaatacatacttaaatgttgtagctgcgtacaccaaataaccgccggttgtatgaagcacaccttttggcttgccggtggaacctgaagtgtacagaataaatagaggatcttcagcattcatagcttccggttgatgctctggggatgctttctcaatcaaatctctccaccacagatccctaccttcttgccaatctatgtcactaccggttcttttcaaaacgattacgtgttcaacagaagttacatttggattctttaacgcatcatcgacattctttttcaatgggatagacctgcctgctcttactccttcgtctgctgtaataactaggcgactactactatctatgatcctgccagctacagcctcgggtgaaaaaccaccgaaaatcacggaatgtacagctcctatccgagcgcatgctagcatcgctacggcggcctctggaaccataggcatataaatagcaacaacgtcaccctttttaatacctaagtctagtaatgtgtttgcgaacctgcacacatctctgtgtagctctctgtacgagatgtgcttggattgtgatgtatcatctccttcccatattatggcggttctatctccgttctcttgtaaatggcggtctaggcaatttgcagccaaattcaatgttccatcttcataccatttaatactgacatttccaggtgcaaatgaggtgtttttaaccttctgatagggtgtgatccaatccagaatcttaccctgctctccccaaaacgtgtcaggatcattgatagattgcttatattttgtctcatattgttcgggattaattaagcaccgatctgctatattagcaggaattgcatgtttgtgggtctggctcat

SEQ ID NO: 16 - protein, 652 aa, Artificial sequence
  Synthetic
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGAVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKPLEEKQAIAMPS

SEQ ID NO: 17 - DNA, 1512 bp, Saccharomyces cerevisiae
atgaggaagctaaatccagctttagaatttagagactttatccaggtcttaaaagatgaagatgacttaatcgaaattaccgaagagattgatccaaatctcgaagtaggtgcaattatgaggaaggcctatgaatcccacttaccagccccgttatttaaaaatctcaaaggtgcttcgaaggatcttttcagcattttaggttgcccagccggtttgagaagtaaggagaaaggagatcatggtagaattgcccatcatctggggctcgacccaaaaacaactatcaaggaaatcatagattatttgctggagtgtaaggagaaggaacctctccccccaatcactgttcctgtgtcatctgcaccttgtaaaacacatatactttctgaagaaaaaatacatctacaaagcctgccaacaccatatctacatgtttcagacggtggcaagtacttacaaacgtacggaatgtggattcttcaaactccagataaaaaatggactaattggtcaattgctagaggtatggttgtagatgacaagcatatcactggtctggtaattaaaccacaacatattagacaaattgctgactcttgggcagcaattggaaaagcaaatgaaattcctttcgcgttatgttttggcgttcccccagcagctattttagttagttccatgccaattcctgaaggtgtttctgaatcggattatgttggcgcaatcttgggtgagtcggttccagtagtaaaatgtgagaccaacgatttaatggttcctgcaacgagtgagatggtatttgagggtactttgtccttaacagatacacatctggaaggcccatttggtgagatgcatggatatgttttcaaaagccaaggtcatccttgtccattgtacactgtcaaggctatgagttacagagacaatgctattctacctgtttcgaaccccggtctttgtacggatgagacacataccttgattggttcactagtggctactgaggccaaggagctggctattgaatctggcttgccaattctggatgcctttatgccttatgaggctcaggctctttggcttatcttaaaggtggatttgaaagggctgcaagcattgaagacaacgcctgaagaattttgtaagaaggtaggtgatatttactttaggacaaaagttggttttatagtccatgaaataattttggtggcagatgatatcgacatatttaacttcaaagaagtcatctgggcctacgttacaagacatacacctgttgcagatcagatggcttttgatgatgtcacttcttttcctttggctccctttgtttcgcagtcatccagaagtaagactatgaaaggtggaaagtgcgttactaattgcatatttagacagcaatatgagcgcagttttgactacataacttgtaattttgaaaagggatatccaaaaggattagttgacaaagtaaatgaaaattggaaaaggtacggatataaataa

SEQ ID NO: 18 - protein, 503 aa, Saccharomyces cerevisiae
MRKLNPALEFRDFIQVLKDEDDLIEITEEIDPNLEVGAIMRKAYESHLPAPLFKNLKGASKDLFSILGCPAGLRSKEKGDHGRIAHHLGLDPKTTIKEIIDYLLECKEKEPLPPITVPVSSAPCKTHILSEEKIHLQSLPTPYLHVSDGGKYLQTYGMWILQTPDKKWTNWSIARGMVVDDKHITGLVIKPQHIRQIADSWAAIGKANEIPFALCFGVPPAAILVSSMPIPEGVSESDYVGAILGESVPVVKCETNDLMVPATSEMVFEGTLSLTDTHLEGPFGEMHGYVFKSQGHPCPLYTVKAMSYRDNAILPVSNPGLCTDETHTLIGSLVATEAKELAIESGLPILDAFMPYEAQALWLILKVDLKGLQALKTTPEEFCKKVGDIYFRTKVGFIVHEIILVADDIDIFNFKEVIWAYVTRHTPVADQMAFDDVTSFPLAPFVSQSSRSKTMKGGKCVTNCIFRQQYERSFDYITCNFEKGYPKGLVDKVNENWKRYGYK

SEQ ID NO: 19 - DNA, 729 bp, Saccharomyces cerevisiae
atgctcctatttccaagaagaactaatatagcctttttcaaaacaacaggcatttttgctaattttcctttgctaggtagaaccattacaacttcaccatctttccttacacataaactgtcaaaggaagtaaccagggcatcaacttcgcctccaagaccaaagagaattgttgtcgcaattactggtgcgactggtgttgcactgggaatcagacttctacaagtgctaaaagagttgagcgtagaaacccatttggtgatttcaaaatggggtgcagcaacaatgaaatatgaaacagattgggaaccgcatgacgtggcggccttggcaaccaagacatactctgttcgtgatgtttctgcatgcatttcgtccggatctttccagcatgatggtatgattgttgtgccctgttccatgaaatcactagctgctattagaatcggttttacagaggatttaattacaagagctgccgatgtttcgattaaagagaatcgtaagttactactggttactcgggaaacccctttatcttccatccatcttgaaaacatgttgtctttatgcagggcaggtgttataatttttcctccggtacctgcgttttatacaagacccaagagccttcatgacctattagaacaaagtgttggcaggatcctagactgctttggcatccacgctgacacttttcctcgttgggaaggaataaaaagcaagtaa

SEQ ID NO: 20 - protein, 242 aa, Saccharomyces cerevisiae
MLLFPRRTNIAFFKTTGIFANFPLLGRTITTSPSFLTHKLSKEVTRASTSPPRPKRIVVAITGATGVALGIRLLQVLKELSVETHLVISKWGAATMKYETDWEPHDVAALATKTYSVRDVSACISSGSFQHDGMIVVPCSMKSLAAIRIGFTEDLITRAADVSIKENRKLLLVTRETPLSSIHLENMLSLCRAGVIIFPPVPAFYTRPKSLHDLLEQSVGRILDCFGIHADTFPRWEGIKSK

SEQ ID NO: 21 - DNA, 1908 bp, Saccharomyces cerevisiae
atggcacctgttacaattgaaaagttcgtaaatcaagaagaacgacaccttgtttccaaccgatcagcaacaattccgtttggtgaatacatatttaaaagattgttgtccatcgatacgaaatcagttttcggtgttcctggtgacttcaacttatctctattagaatatctctattcacctagtgttgaatcagctggcctaagatgggtcggcacgtgtaatgaactgaacgccgcttatgcggccgacggatattcccgttactctaataagattggctgtttaataaccacgtatggcgttggtgaattaagcgccttgaacggtatagccggttcgttcgctgaaaatgtcaaagttttgcacattgttggtgtggccaagtccatagattcgcgttcaagtaactttagtgatcggaacctacatcatttggtcccacagctacatgattcaaattttaaagggccaaatcataaagtatatcatgatatggtaaaagatagagtcgcttgctcggtagcctacttggaggatattgaaactgcatgtgaccaagtcgataatgttatccgcgatatttacaagtattctaaacctggttatatttttgttcctgcagattttgcggatatgtctgttacatgtgataatttggttaatgttccacgtatatctcaacaagattgtatagtatacccttctgaaaaccaattgtctgacataatcaacaagattactagttggatatattccagtaaaacacctgcgatccttggagacgtactgactgataggtatggtgtgagtaactttttgaacaagcttatctgcaaaactgggatttggaatttttccactgttatgggaaaatctgtaattgatgagtcaaacccaacttatatgggtcaatataatggtaaagaaggtttaaaacaagtctatgaacattttgaactgtgcgacttggtcttgcattttggagtcgacatcaatgaaattaataatgggcattatacttttacttataaaccaaatgctaaaatcattcaatttcatccgaattatattcgccttgtggacactaggcagggcaatgagcaaatgttcaaaggaatcaattttgcccctattttaaaagaactatacaagcgcattgacgtttctaaactttctttgcaatatgattcaaatgtaactcaatatacgaacgaaacaatgcggttagaagatcctaccaatggacaatcaagcattattacacaagttcacttacaaaagacgatgcctaaatttttgaaccctggtgatgttgtcgtttgtgaaacaggctcttttcaattctctgttcgtgatttcgcgtttccttcgcaattaaaatatatatcgcaaggatttttcctttccattggcatggcccttcctgccgccctaggtgttggaattgccatgcaagaccactcaaacgctcacatcaatggtggcaacgtaaaagaggactataagccaagattaattttgtttgaaggtgacggtgcagcacagatgacaatccaagaactgagcaccattctgaagtgcaatattccactagaagttatcatttggaacaataacggctacactattgaaagagccatcatgggccctaccaggtcgtataacgacgttatgtcttggaaatggaccaaactatttgaagcattcggagacttcgacggaaagtatactaatagcactctcattcaatgtccctctaaattagcactgaaattggaggagcttaagaattcaaacaaaagaagcgggatagaacttttagaagtcaaattaggcgaattggatttccccgaacagctaaagtgcatggttgaagcagcggcacttaaaagaaataaaaaatag

SEQ ID NO: 22 - DNA, 1692 bp, Saccharomyces cerevisiae
atgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtcaaggactacaaacctgttgctgtcccagctagagttccaattaccaagtctactccagctaacactccaatgaagcaagaatggatgtggaaccatttgggtaacttcttgagagaaggtgatattgttattgctgaaaccggtacttccgccttcggtattaaccaaactactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaataa